(19) United States
(12) Patent Application Publication
(10) Pub. No.: US 2004/0197888 A1
(43) Pub. Date: Oct. 7, 2004

(54) Alternatively spliced isoforms of histone deacetylase 3 (HDAC3)

(76) Inventors: Christopher D. Armour, Kirkland, WA (US); Patrick M. Loerch, Brookline, MA (US); John C. Castle, Seattle, WA (US); Jason M. Johnson, Seattle, WA (US)

Correspondence Address: R. Douglas Bradley, Merck & Co., Inc., Patent Department RY60-30, P.O. Box 2000, Rahway, NJ, 07065-0907, US

(21) Appl. No.: 10/745,242

(22) Filed: Dec. 22, 2003

Related U.S. Application Data
(60) Provisional application No. 60/437,666, filed on Dec. 31, 2002.
(60) Provisional application No. 60/478,233, filed on Jun. 12, 2003.

(51) Int. Cl. C12N 9/18; C07H 21/04

(52) U.S. Cl. 435/197; 435/69.1; 435/320.1; 435/325; 536/23.2

(57) ABSTRACT

The present invention features nucleic acids and polypeptides encoding four novel splice variant isoforms of histone deacetylase 3 (HDAC3). The polynucleotide sequences of HDAC3sv1.1, HDAC3sv1.2, HDAC3sv2, HDAC3sv3, HDAC3sv4, HDAC3sv5, and HDAC3sv6 are provided by SEQ ID NO 1, SEQ ID NO 3, SEQ ID NO 5, SEQ ID NO 7, SEQ ID NO 9, SEQ ID NO 20, and SEQ ID NO 21, respectively. The amino acid sequences for HDAC3sv1.1, HDAC3sv1.2, HDAC3sv2, HDAC3sv3, and HDAC3sv4 are provided by SEQ ID NO 2, SEQ ID NO 4, SEQ ID NO 6, SEQ ID NO 8, and SEQ ID NO 10, respectively. The present invention also provides methods for using HDAC3sv1.1, HDAC3sv1.2, HDAC3sv2, HDAC3sv3, and HDAC3sv4 polynucleotides and proteins to screen for compounds that bind to HDAC3sv1.1, HDAC3sv1.2, HDAC3sv2, HDAC3sv3, or HDAC3sv4, respectively.

This application claims priority to U.S. Provisional Patent Application Serial No. 06/437,666 filed on Dec. 31, 2002, and U.S. Provisional Patent Application Serial No. 06/478,233 filed on Jun. 12, 2003, which are both incorporated by reference herein in their entirety.

### BACKGROUND OF THE INVENTION

The references cited herein are not admitted to be prior art to the claimed invention.

The DNA in an eukaryotic cell is compacted 50,000-fold by association with a group of proteins called histones. The compacted DNA-histone complex is called chromatin. The basic unit of chromatin is a nucleosome, which comprises about 146 base pairs of DNA tightly wrapped around a core of two copies each of four different histone proteins (termed H2A, H2B, H3 and H4), a linker histone (H1 or H5) and a variable length of linker DNA (Duggan and Thomas, 2000, J. Mol. Biol. 304:21-33). The positively charged side chains of the histones interact with the negatively charged phosphates in the DNA (Luger et al, 1997, Nature 389, 251-260), while the H1 and H5 linker histones function to stabilize the condensed chromatin structure and to aid in further compaction of DNA (Carruthers and Hansen, 2000, J. Biol. Chem. 275, 37285-37290). The highly organized compact structure of the chromatin restricts the access of proteins such as transcription factors to the DNA.

Histones are posttranslationally modified by a number of mechanisms that include phosphorylation, acetylation and deacetylation (Davie, J. R., 1998, Curr. Opin. in Dev. Biol. 8, 173-178; Strahl, B. D. & Allis., C. D., 2000, Nature 403, 41-45). The most abundant covalent modification of histones is the reversible acetylation of H3 and H4. The amount of acetylation is modulated by two classes of enzymes, histone acetylases (HATs) and histone deacetylases (HDACs). Substrates for these enzymes include the ε-amino groups of lysines of N-terminal tails of histones. Acetylation decreases chromatin compaction thereby increasing transcription factor accessibility. In contrast, deacetylation has the opposite effect on chromatin structure, leading to greater compaction of chromatin, thereby reducing transcription factor accessibility to nucleosomal DNA (Wolfe, 1998, Chromatin Structure and Function, Third Edition, Academic Press, San Diego). Prototypical examples of transcription factors whose transcriptional activity is facilitated by histone deacetylation, are transcription factors involved in activation of the retinoid/steroid superfamily of receptors. Thus, histone acetylation-deacetylation reactions modulate gene activity by changing nucleosome structure.

A growing number of human HDACs have been identified in various biological systems (Gray and Eckstrom, Exp. Cell Res. 262, 75-83). Human HDAC proteins are divided into three classes based on sequence similarity to yeast HDAC homologs: the yeast RPD3-related histone deacetylases (class I), which includes HDAC-3; the HAD-1-like deacetylases (class II), which share domain homology with DDAC-1, and the SIR2-like deacetylases (class III), which are NAD⁺-dependent for enzymatic activity.

Several structural classes of HDAC inhibitors have been identified. These include the following: 1) short-chain fatty acids (e.g., butyrates); 2) hydroxamic acids (e.g., trichostatin A (TSA), suberoylanilide hydroxamic acid, and oxamflatin); 3) cyclic tetrapeptides containing a 2-amino-8-oxo-9,10-epoxy-decanoyl (AOE) moiety (e.g., trapoxin A); 4) cyclic peptides not containing the AOE moiety (e.g., FR901228 and apicidin); and 5) benzamides (e.g., MS-27-275) (Kramer et al., 2001, Trends in Endocrinol. & Metabol. 12, 294-300; Marks et al., 2000, Journal of the Natl. Cancer Institute 92, 1210-1216). These compounds have been disclosed to have anti-cancer properties in laboratory model systems (Kramer et al., 2001, Trends in Endocrinol & Metabol. 12, 294-300; Marks et al., 2000, Journal of the Natl. Cancer Institute 92, 1210-1216). In addition, antisense oligonucleotides have been used as inhibitors of HDAC3 activity (U.S. patent application No. 20020061860). Among these HDAC3 inhibitors, hydroxamic-based compounds and depudecin inhibit deacetylase activity at micromolar concentrations, whereas TSA inhibits HDAC activity at nanomolar concentrations by binding to HDAC enzymes. HDAC inhibitors cause the accumulation of acetylated histones in cancer cells and in tumor cells. The build-up of acetylated histones in cancer cells is thought to relax chromatin structure, thereby allowing the expression of genes that inhibit tumor cell growth and enhance cell death. Because of their anti-proliferative activities and their ability to induce apoptosis, HDAC inhibitors have been used as anticancer agents particularly for chemotherapy in clinical trials (Marks et al., 2000, Natl. Cancer. Inst. 92, 1210-1216). Chromatin repression due to defects in mammalian HDAC activities has been associated with numerous forms of cancer, in particular acute promyelocytic leukemia and non-Hodgkin's lymphomas (Kramer, et al., 2001, Trends in Endocrinol. & Metabol. 12, 294-300).

The human HDAC3 gene has been mapped to chromosome 5, locus q31. A number of disease phenotypes, such as asthma, inherited deafness, congenital leukemia, large-cell lymphoma, myelodysplastic syndrome, have been mapped to this chromosome region (Randhava et al., 1998, Genomics 51, 262-269).

Recently, human histone acetylation deficiency has been associated with Huntington's disease, Kennedy's disease, spino cerebellar ataxis and dentorubral pallidoluysian atrophy (Zoghibi and Orr, 2000, Ann. Rev. Neurosci. 23, 217-247; Hughes, 2002, Curr. Biol. 12, R141-R143). These diseases are associated with expanded numbers of glutamine residues (polyQ) in some proteins as a consequence of the presence of CAG triplet repeats (CAG codes for glutamine) in corresponding gene coding sequences. PolyQ peptide domains form insoluble protein aggregates, and if present in critical metabolic enzymes, results in their complete inactivation. Such is the case with histone acetylation enzyme—the CREB-binding protein (CBP), a histone acetyltransferase. In Huntington's patients, CBP acetyltransferase is sequestered in an inactive state by the formation of inclusion bodies with polyQ-containing proteins, thereby resulting in a histone hypoacetylation defect. Since histone acetylation is controlled by the balance between acetylation and deacetylation, and since there are no small molecule drugs that can enhance acetylation, one therapeutic strategy is to suppress deacetylation using inhibitors, such as trichostatin (see above), which results in increased acetylation. This strategy has been successfully demonstrated in model systems of Huntington and Kennedy diseases (Steffan et al., 2001, Nature 413, 739-743; Hughes, 2001, Proc. Natl. Acad. Sci., USA. 98, 13201-13206; McCampbell., 2001, Proc. Natl. Acad. Sci. USA. 98, 15179-15184; Tylor and Fischbeck, 2002, Trends Mol. Med. 8, 195-197), and is currently being viewed as viable strategy for treating patients with cancer and poly-glutamine diseases (Marks et al., 2001, Curr. Opin. Oncol. 13, 477-483; Hughes, 2002, Curr. Biol. 12, R141-R143).

Because of the multiple therapeutic values of drugs targeting the HDAC3 protein (Kramer et al., 2001, Trends in Endocrinol. & Metabol. 12, 294-300), there is a need in the art for compounds that selectively bind to isoforms of human HDAC3. The present invention is directed towards novel HDAC3 isoforms (HDAC3sv1.1, HDAC3sv1.2, HDAC3sv2, HDAC3sv3, and HDAC3sv4) and uses thereof.

### SUMMARY OF THE INVENTION

Microarray experiments and RT-PCR have been used to identify and confirm the presence of novel splice variants of human HDAC3 mRNA. More specifically, the present invention features polynucleotides encoding different protein isoforms of HDAC3. A polynucleotide sequence encoding HDAC3sv1.1 is provided by SEQ ID NO 1. An amino acid sequence for HDAC3sv1.1 is provided by SEQ ID NO 2. A polynucleotide sequence encoding HDAC3sv1.2 is provided by SEQ ID NO 3. An amino acid sequence for HDAC3sv1.2 is provided by SEQ ID NO 4. A polynucleotide sequence encoding HDAC3sv2 is provided by SEQ ID NO 5. An amino acid sequence for HDAC3sv2 is provided by SEQ ID NO 6. A polynucleotide sequence encoding HDAC3sv3 is provided by SEQ ID NO 7. An amino acid sequence for HDAC3sv3 is provided by SEQ ID NO 8. A polynucleotide sequence encoding HDAC3sv4 is provided by SEQ ID NO 9. An amino acid sequence for HDAC3sv4 is provided by SEQ ID NO 10.

Thus, a first aspect of the present invention describes a purified HDAC3sv1.1 encoding nucleic acid, a purified HDAC3sv1.2 encoding nucleic acid, a purified HDAC3sv2 encoding nucleic acid, a purified HDAC3sv3 encoding nucleic acid, and a purified HDAC3sv4 encoding nucleic acid. The HDAC3sv1.1 encoding nucleic acid comprises SEQ ID NO 1 or the complement thereof. The HDAC3sv1.2 encoding nucleic acid comprises SEQ ID NO 3 or the complement thereof. The HDAC3sv2 encoding nucleic acid comprises SEQ ID NO 5 or the complement thereof. The HDAC3sv3 encoding nucleic acid comprises SEQ ID NO 7 or the complement thereof. The HDAC3sv4 encoding nucleic acid comprises SEQ ID NO 9 or the complement thereof. Reference to the presence of one region does not indicate that another region is not present. For example, in different embodiments the inventive nucleic acid can comprise, consist, or consist essentially of an encoding nucleic acid sequence of SEQ ID NO 1, can comprise, consist, or consist essentially of the nucleic acid sequence of SEQ ID NO 3, can comprise, consist, or consist essentially of the nucleic acid sequence of SEQ ID NO 5, can comprise, consist, or consist essentially of the nucleic acid sequence of SEQ ID NO 7, or alternatively can comprise, consist, or consist essentially of the nucleic acid sequence of SEQ ID NO 9.

Another aspect of the present invention describes a purified HDAC3sv1.1 polypeptide that can comprise, consist or consist essentially of the amino acid sequence of SEQ ID NO 2. An additional aspect describes a purified HDAC3sv1.2 polypeptide that can comprise, consist, or consist essentially of the amino acid sequence of SEQ ID NO 4. An additional aspect describes a purified HDAC3sv2 polypeptide that can comprise, consist, or consist essentially of the amino acid sequence of SEQ ID NO 6. An additional aspect describes a purified HDAC3sv3 polypeptide that can comprise, consist, or consist essentially of the amino acid sequence of SEQ ID NO 8. An additional aspect describes a purified HDAC3sv4 polypeptide that can comprise, consist, or consist essentially of the amino acid sequence of SEQ ID NO 10.

Another aspect of the present invention describes expression vectors. In one embodiment of the invention, the inventive expression vector comprises a nucleotide sequence encoding a polypeptide comprising, consisting, or consisting essentially of SEQ ID NO 2, wherein the nucleotide sequence is transcriptionally coupled to an exogenous promoter. In other embodiments, the inventive expression vector comprises a nucleotide sequence encoding a polypeptide comprising, consisting, or consisting essentially of SEQ ID NO 4, SEQ ID NO 6, SEQ ID NO 8, or SEQ ID NO 10, wherein the nucleotide sequence is transcriptionally coupled to an exogenous promoter.

Alternatively, the nucleotide sequence comprises, consists, or consists essentially of SEQ ID NO 1, and is transcriptionally coupled to an exogenous promoter. In other embodiments, the nucleotide sequence comprises, consists, or consists essentially of SEQ ID NO 3, SEQ ID NO 5, SEQ ID NO 7, or SEQ ID NO 9, wherein the sequence is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention, describes a recombinant cell made by a process comprising the step of introducing into the cell an expression vector comprising a nucleotide sequence comprising, consisting, or consisting essentially of SEQ ID NO 1, SEQ ID NO 3, SEQ ID NO 5, SEQ ID NO 7, or SEQ ID NO 9, or a nucleotide sequence encoding a polypeptide comprising, consisting, or consisting essentially of an amino acid sequence of SEQ ID NO 2, SEQ ID NO 4, SEQ ID NO 6, SEQ ID NO 8, or SEQ ID NO 10, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing HDAC3sv1.1, HDAC3sv1.2, HDAC3sv2, HDAC3sv3, or HDAC3sv4 polypeptide comprising SEQ ID NO 2, SEQ ID NO 4, SEQ ID NO 6, SEQ ID NO 8, or SEQ ID NO 10, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to HDAC3sv1.1 as compared to one or more HDAC3 isoform polypeptides that are not HDAC3sv1.1. In other embodiments, a purified antibody preparation is provided comprising antibody that binds selectively to HDAC3sv1.2, HDAC3sv2, HDAC3sv3, or HDAC3sv4 as compared to one or more different HDAC3 isoform polypeptides that are not the respective HDAC3 isoform polypeptide.

Another aspect of the present invention provides a method of screening for a compound that binds to HDAC3sv1.1, HDAC3sv1.2, HDAC3sv2, HDAC3sv3, HDAC3sv4, or fragments thereof. In one embodiment, the method comprises the steps of: (a) expressing a polypeptide comprising the amino acid sequence of SEQ ID NO 2 or a fragment thereof from recombinant nucleic acid; (b) providing to said polypeptide a labeled HDAC3 ligand that binds to said polypeptide and a test preparation comprising one or more test compounds; (c) and measuring the effect of said test preparation on binding of said test preparation to said polypeptide comprising SEQ ID NO 2. Alternatively, this method could be performed using SEQ ID NO 4, SEQ ID NO 6, SEQ ID NO 8, or SEQ ID NO 10, in place of SEQ ID NO 2.

In another embodiment of the method, a compound is identified that binds selectively to HDAC3sv1.1 polypeptide as compared to one or more HDAC3 isoform polypeptides that are not HDAC3sv1.1. This method comprises the steps of: providing a HDAC3sv1.1 polypeptide comprising SEQ ID NO 2; providing a HDAC3 isoform polypeptide that is not HDAC3sv1.1, contacting said HDAC3sv1.1 polypeptide and said HDAC3 isoform polypeptide that is not HDAC3sv1.1 with a test preparation comprising one or more test compounds; and determining the binding of said test preparation to said HDAC3sv1.1 polypeptide and to HDAC3 isoform polypeptide that is not HDAC3sv1.1, wherein a test preparation that binds to said HDAC3sv1.1 polypeptide but does not bind to said HDAC3 isoform polypeptide that is not HDAC3sv1.1 contains a compound that selectively binds said HDAC3sv1.1 polypeptide. Alternatively, the same method can be performed using HDAC3sv1.2 polypeptide comprising, consisting, or consisting essentially of SEQ ID NO 4. Alternatively, the same method can be performed using HDAC3sv2 polypeptide comprising, consisting, or consisting essentially of SEQ ID NO 6. Alternatively, the same method can be performed using HDAC3sv3 polypeptide comprising, consisting, or consisting essentially of SEQ ID NO 8. Alternatively, the same method can be performed using HDAC3sv4 polypeptide comprising, consisting, or consisting essentially of SEQ ID NO 10.

In another embodiment of the invention, a method is provided for screening for a compound able to bind to or interact with a HDAC3sv1.1 protein or a fragment thereof comprising the steps of: expressing a HDAC3sv1.1 polypeptide comprising SEQ ID NO 2 or a fragment thereof from a recombinant nucleic acid; providing to said polypeptide a labeled HDAC3 ligand that binds to said polypeptide and a test preparation comprising one or more compounds; and measuring the effect of said test preparation on binding of said labeled HDAC3 ligand to said polypeptide, wherein a test preparation that alters the binding of said labeled HDAC3 ligand to said polypeptide contains a compound that binds to or interacts with said polypeptide. In an alternative embodiment, the method is performed using HDAC3sv1.2 polypeptide comprising, consisting, or consisting essentially of SEQ ID NO 4 or a fragment thereof. In an alternative embodiment, the method is performed using HDAC3sv2 polypeptide comprising, consisting, or consisting essentially of SEQ ID NO 6 or a fragment thereof. In an alternative embodiment, the method is performed using HDAC3sv3 polypeptide comprising, consisting, or consisting essentially of SEQ ID NO 8 or a fragment thereof. In an alternative embodiment, the method is performed using HDAC3sv4 polypeptide comprising, consisting, or consisting essentially of SEQ ID NO 10 or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE FIGURES

FIG. 1A illustrates the exon structure of HDAC3 mRNA corresponding to the known reference form of HDAC3 mRNA (labeled NM_{—}003883) and the exon structure corresponding to splice variants described herein (labeled HDAC3sv1, HDAC3sv2, HDAC3sv3, HDAC3sv4, HDAC3sv5, and HDAC3sv6, respectively). FIG. 1B depicts the nucleotide sequences of the exon junctions resulting from the splicing of exon 2 to exon 7 in the case of HDAC3sv1mRNA; the splicing of exon 2 to exon 5 in the case of the HDAC3sv2 mRNA; the splicing of exon 2 to exon 4 in the case of HDAC3sv3 mRNA; the splicing of exon 4 to intron 4 and intron 4 to exon 5 in the case of HDAC3sv4mRNA; the splicing of exon 4 to intron 4, intron 4 to exon 5, exon 5 to intron 5, and intron 5 to exon 6 in the case of the HDAC3sv5 mRNA; and the splicing of exon 2 to exon 4, of exon 5 to intron 5, of intron 5 to exon 6, of exon 10 to exon 12, and of exon 12 to exon 14, in the case of HDAC3sv6 mRNA.

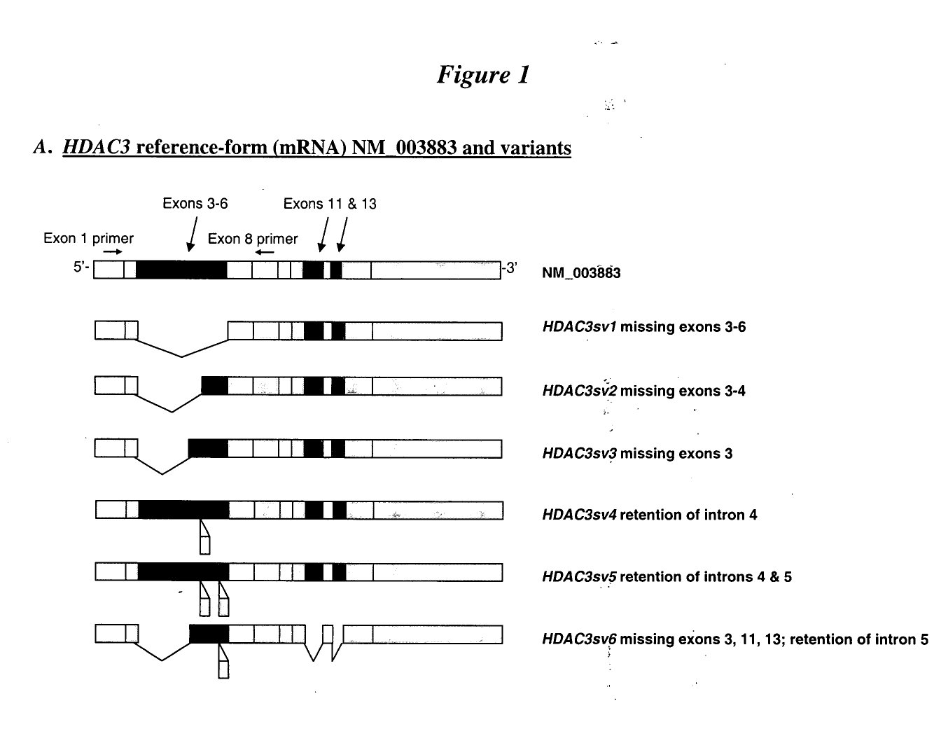

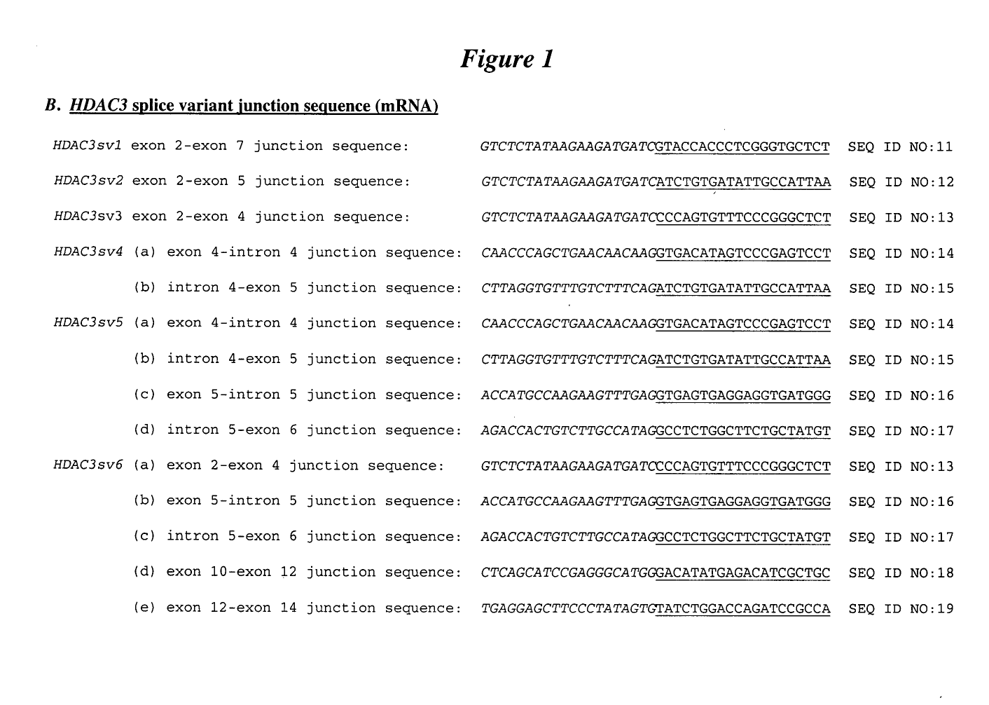

In FIG. 1B, in the case of HDAC3sv1, the nucleotides shown in italics represent the 20 nucleotides at the 3′ end of exon 2 and the nucleotides shown in underline represent the 20 nucleotides at the 5′ end of exon 7 [SEQ ID NO 11]. In the case of HDAC3sv2, the nucleotides shown in italics represent the 20 nucleotides at the 3′ end of exon 2 and the nucleotides shown in underline represent the 20 nucleotides at the 5′ end of exon 5 [SEQ ID NO 12]. In the case of HDAC3sv3, the nucleotides shown in italics represent the 20 nucleotides at the 3′ end of exon 2 and the nucleotides shown in underline represent the 20 nucleotides at the 5′ end of exon 4 [SEQ ID NO 13]. In the case of HDAC3sv4, in (a) the nucleotides shown in italics represent the 20 nucleotides at the 3′ end of exon 4 and the nucleotides shown in underline represent the 20 nucleotides at the 5′ end of intron 4 [SEQ ID NO 14], and in (b) the nucleotides shown in italics represent the 20 nucleotides at the 3′ end of intron 4 and the nucleotides shown in underline represent the 20 nucleotides at the 5′ end of exon 5 [SEQ ID NO 15]. In the case of HDAC3sv5, in (a) the nucleotides shown in italics represent the 20 nucleotides at the 3′ end of exon 4 and the nucleotides shown in underline represent the 20 nucleotides at the 5′ end of intron 4 [SEQ ID NO 14], in (b) the nucleotides shown in italics represent the 20 nucleotides at the 3′ end of intron 4 and the nucleotides shown in underline represent the 20 nucleotides at the 5′ end of exon 5 [SEQ ID NO 15], in (c) the nucleotides shown in italics represent the 20 nucleotides at the 3′ end of exon 5 and the nucleotides shown in underline represent the 20 nucleotides at the 5′ end of intron 5 [SEQ ID NO 16], and in (d) the nucleotides shown in italics represent the 20 nucleotides at the 3′ end of intron 5 and the nucleotides shown in underline represent the 20 nucleotides at the 5′ end of exon 6 [SEQ ID NO 17]. In the case of HDAC3sv6, in (a) the nucleotides shown in italics represent the 20 nucleotides at the 3′ end of exon 2 and the nucleotides shown in underline represent the 20 nucleotides at the 5′ end of exon 4 [SEQ ID NO 13], in (b) the nucleotides shown in italics represent the 20 nucleotides at the 3′ end of exon 5 and the nucleotides shown in underline represent the 20 nucleotides at the 5′ end of intron 5 [SEQ ID NO 16], in (c) the nucleotides shown in italics represent the 20 nucleotides at the 3′ end of intron 5 and the nucleotides shown in underline represent the 20 nucleotides at the 5′ end of exon 6 [SEQ ID NO 17], in (d) the nucleotides shown in italics represent the 20 nucleotides at the 3′ end of exon 10 and the nucleotides shown in underline represent the 20 nucleotides at the 5′ end of exon 12 [SEQ ID NO 18], and in (e) the nucleotides shown in italics represent the 20 nucleotides at the 3′ end of exon 12 and the nucleotides shown in underline represent the 20 nucleotides at the 5′ end of exon 14 [SEQ ID NO 19].

### DEFINITIONS

Unless defined otherwise, all technical and scientific terms used herein have the meaning commonly understood by one of ordinary skill in the art to which this invention belongs.

As used herein, “HDAC3” refers to a histone deacetylase protein (NP_{—}003874). In contrast, reference to an HDAC3 isoform, includes NP_{—}003874 and other polypeptide isoform variants of HDAC3.

As used herein, “HDAC3sv1.1”, “HDAC3sv1.2”, “HDAC3sv2”, “HDAC3sv3”, and HDAC3sv4” refer to splice variant isoforms of human HDAC3 protein, wherein the splice variant isoforms have the amino acid sequence set forth in SEQ ID NO 2 (for HDAC3sv1.1), SEQ ID NO 4 (for HDAC3sv1.2), SEQ ID NO 6 (for HDAC3sv2), SEQ ID NO 8 (for HDAC3sv3), and SEQ ID NO 10 (for HDAC3sv4).

As used herein, “HDAC3” refers to polynucleotides encoding HDAC3.

As used herein, “HDAC3sv1” refers to polynucleotides that are identical to HDAC3 encoding polynucleotides, except that the sequences represented by exons 3, 4, 5 and 6 of the HDAC3 messenger RNA are not present in HDAC3sv1.

As used herein, “HDAC3sv1.1” refers to polynucleotides encoding HDAC3sv1.1 having an amino acid sequence set forth in SEQ ID NO 2. As used herein, “HDAC3sv1.2” refers to polynucleotides encoding HDAC3sv1.2 having an amino acid sequence set forth in SEQ ID NO 4. As used herein, “HDAC3sv2” refers to polynucleotides encoding HDAC3sv2 having an amino acid sequence set forth in SEQ ID NO 6. As used herein, “HDAC3sv3” refers to polynucleotides encoding HDAC3sv3 having an amino acid sequence set forth in SEQ ID NO 8. As used herein, “HDAC3sv4” refers to polynucleotides encoding HDAC3sv4 having an amino acid sequence set forth in SEQ ID NO 10. As used herein, “HDAC3sv5” refers to a polynucleotide sequence set forth in SEQ ID NO 20 encoding HDAC3sv4 having an amino acid sequence set forth in SEQ ID NO 10. As used herein, “HDAC3sv6” refers to a polynucleotide sequence set forth in SEQ ID NO 21 encoding HDAC3sv3 having an amino acid sequence set forth in SEQ ID NO 8.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human HDAC3sv1.1, HDAC3sv1.2, HDAC3sv2, HDAC3sv3, and HDAC3sv4 that are alternatively spliced isoforms of HDAC3, and to the amino acid sequences encoding these proteins. SEQ ID NO 1, SEQ ID NO 3, SEQ ID NO 5, SEQ ID NO 7, and SEQ ID NO 9 are polynucleotide sequences representing exemplary open reading frames that encode the HDAC3sv1.1, HDAC3sv1.2, HDAC3sv2, HDAC3sv3, and HDAC3sv4 proteins, respectively. SEQ ID NO 2 shows the polypeptide sequence of HDAC3sv1.1. SEQ ID NO 4 shows the polypeptide sequence of HDAC3sv1.2. SEQ ID NO 6 shows the polypeptide sequence of HDAC3sv2. SEQ ID NO 8 shows the polypeptide sequence of HDAC3sv3. SEQ ID NO 10 shows the polypeptide sequence of HDAC3sv4.

HDAC3sv1.1, HDAC3sv1.2, HDAC3sv2, HDAC3sv3, and HDAC3sv4 polynucleotide sequences encoding HDAC3sv1.1, HDAC3sv1.2, HDAC3sv2, HDAC3sv3, and HDAC3sv4 proteins, as exemplified and enabled herein, include a number of specific, substantial and credible utilities. For example, HDAC3sv1.1, HDAC3sv1.2, HDAC3sv2, HDAC3sv3, and HDAC3sv4 encoding nucleic acids were identified in an mRNA sample obtained from a human source (see Example 1). Such nucleic acids can be used as hybridization probes to distinguish between cells that produce HDAC3sv1.1, HDAC3sv1.2, HDAC3sv2, HDAC3sv3, HDAC3sv4, HDAC3sv5, and HDAC3sv6 transcripts from human or non-human cells (including bacteria) that do not produce such transcripts. Similarly, antibodies specific for HDAC3sv1.1, HDAC3sv1.2, HDAC3sv2, HDAC3sv3, and HDAC3sv4 can be used to distinguish between cells that express HDAC3sv1.1, HDAC3sv1.2, HDAC3sv2, HDAC3sv3, or HDAC3sv4 from human or non-human cells (including bacteria) that do not express HDAC3sv1.1, HDAC3sv1.2, HDAC3sv2, HDAC3sv3, or HDAC3sv4.

HDAC3 is an important drug target for the management of cancer chemotherapy (Cares & Seto, 2001, J. Cell Physiol. 184, 1-16). Given the potential importance of HDAC3 activity to the therapeutic management of cancer it is of value to identify HDAC3 isoforms and identify HDAC3-ligand compounds that are isoform specific, as well as compounds that are effective ligands for two or more different HDAC3 isoforms. In particular, it may be important to identify compounds that are effective inhibitors of a specific HDAC3 isoform activity, yet does not bind to or interact with a plurality of different HDAC3 isoforms. Compounds that bind to or interact with multiple HDAC3 isoforms may require higher drug doses to saturate multiple HDAC3-isoform binding sites and thereby result in a greater likelihood of secondary non-therapeutic side effects. Furthermore, biological effects could also be caused by the interactions of a drug with the HDAC3sv1.1, HDAC3sv1.2, HDAC3sv2, HDAC3sv3, or HDAC3sv4 isoforms specifically. For the foregoing reasons, HDAC3sv1.1, HDAC3sv1.2, HDAC3sv2, HDAC3sv3, and HDAC3sv4 proteins represent useful compound binding targets and have utility in the identification of new HDAC3-ligands exhibiting a preferred specificity profile and having greater efficacy for their intended use.

In some embodiments, HDAC3sv1.1, HDAC3sv1.2, HDAC3sv2, HDAC3sv3, or HDAC3sv4 activity is modulated by a ligand compound to achieve one or more of the following: prevent or reduce the risk of occurrence, or recurrence of cancers (in particular, acute myeloid leukemia and non-Hodgkin's lymphoma and myelodysplastic syndrome). Compounds that treat cancers are particularly important because of the cause-and-effect relationship between cancers and mortality (National Cancer Institute's Cancer Mortality Rates Registry, http://www3.cancer.gov/atlasplus/charts.html, last visited Dec. 31, 2002).

Compounds modulating HDAC3sv1.1, HDAC3sv1.2, HDAC3sv2, HDAC3sv3, or HDAC3sv4 include agonists, antagonists, and allosteric modulators. While not wishing to be limited to any particular theory of therapeutic efficacy, generally, but not always, HDAC3sv1.1, HDAC3sv1.2, HDAC3sv2, HDAC3sv3, or HDAC3sv4 compounds are used to inhibit deacetylase activity, thereby decreasing transcriptional repression. The inhibition of deacetylase activity has been shown to have therapeutic effects in the treatment of cancer in clinical trials (Kramer et al., 2001, Trends in Endocrinol. & Metabol. 12, 294-300) and in model systems of Huntington disease (Steffan et al., 2001, Nature 413, 739-743). Inhibitors of HDAC3 achieve clinical efficacy by a number of known or unknown mechanisms. In the case of cancer treatment, it is hypothesized that inhibition of deacetylation allows the expression of genes that inhibit tumor cell growth and enhance cell death (Marks et al., 2000, J. Natl. Cancer Inst. 92, 1210-1216). In the case of Huntington disease, the hypothesis is that the decrease in deacetylation increases acetylation of histones, thereby compensating for the hypoacetylation defect present in persons with Huntington disease (Steffan et al., 2001, Nature 413, 739-743).

HDAC3sv1.1, HDAC3sv1.2, HDAC3sv2, HDAC3sv3, or HDAC3sv4 activity can also be affected by modulating the cellular abundance of transcripts encoding HDAC3sv1.1, HDAC3sv1.2, HDAC3sv2, HDAC3sv3, or HDAC3sv4, respectively. Compounds modulating the abundance of transcripts encoding HDAC3sv1.1, HDAC3sv1.2, HDAC3sv2, HDAC3sv3, or HDAC3sv4 include a cloned polynucleotide encoding HDAC3sv1.1, HDAC3sv1.2, HDAC3sv2, HDAC3sv3, or HDAC3sv4, respectively, that can express HDAC3sv1.1, HDAC3sv1.2, HDAC3sv2, HDAC3sv3, or HDAC3sv4 in vivo, antisense nucleic acids and siRNAs targeted to HDAC3sv1, HDAC3sv1.1, HDAC3sv1.2, HDAC3sv2, HDAC3sv3, HDAC3sv4, HDAC3sv5, or HDAC3sv6 transcripts, and enzymatic nucleic acids, such as ribozymes targeted to HDAC3sv1, HDAC3sv1.1, HDAC3sv1.2, HDAC3sv2, HDAC3sv3, HDAC3sv4, HDAC3sv5, or HDAC3sv6 transcripts.

In some embodiments, HDAC3sv1.1, HDAC3sv1.2, HDAC3sv2, HDAC3sv3, or HDAC3sv4 activity is modulated to achieve a therapeutic effect upon diseases in which regulation of histone deacetylation is desirable. For example, acute myeloid leukemia may be treated by modulating HDAC3sv1.1, HDAC3sv1.2, HDAC3sv2, HDAC3sv3, or HDAC3sv4 activities to decrease deacetylation. In other embodiments, Huntington disease may be treated by modulating HDAC3sv1.1, HDAC3sv1.2, HDAC3sv2, HDAC3sv3, or HDAC3sv4 activities to achieve increased levels of histone acetylation by reducing histone deacetylation.

HDAC3sv1.1, HDAC3sv1.2, HDAC3sv2, HDAC3sv3, HDAC3sv4, HDAC3sv5, and HDAC3sv6

Nucleic Acids

HDAC3sv1.1 nucleic acids contain regions that encode for polypeptides comprising, consisting, or consisting essentially of SEQ ID NO 2. HDAC3sv1.2 nucleic acids contain regions that encode for polypeptides comprising, consisting, or consisting essentially of SEQ ID NO 4. HDAC3sv2 nucleic acids contain regions that encode for polypeptides comprising, consisting, or consisting essentially of SEQ ID NO 6. HDAC3sv3 nucleic acids contain regions that encode for polypeptides comprising, consisting, or consisting essentially of SEQ ID NO 8. HDAC3sv4 nucleic acids contain regions that encode for polypeptides comprising, consisting, or consisting essentially of SEQ ID NO 10. HDAC3sv5 nucleic acids contain regions that encode for polypeptides comprising, consisting, or consisting essentially of SEQ ID NO 10. HDAC3sv6 nucleic acids contain regions that encode for polypeptides comprising, consisting, or consisting essentially of SEQ ID NO 8.

The HDAC3sv1.1, HDAC3sv1.2, HDAC3sv2, HDAC3sv3, HDAC3sv4, HDAC3sv5, and HDAC3sv6 nucleic acids have a variety of uses, such as use as a hybridization probe or PCR primer to identify the presence of HDAC3sv1.1, HDAC3sv1.2, HDAC3sv2, HDAC3sv3, HDAC3sv4, HDAC3sv5, or HDAC3sv6 nucleic acids, respectively; use as a hybridization probe or PCR primer to identify nucleic acids encoding for proteins related to HDAC3sv1.1 (encoded for example by HDAC3sv1.1), HDAC3sv1.2 (encoded for example by HDAC3sv1.2), HDAC3sv2 (encoded for example by HDAC3sv2), HDAC3sv3 (encoded for example by HDAC3sv3 or HDAC3sv6), or HDAC3sv4 (encoded for example by HDAC3sv4 or HDAC3sv5); and/or use for recombinant expression of HDAC3sv1.1 (encoded for example by HDAC3sv1.1), HDAC3sv1.2 (encoded for example by HDAC3sv1.2), HDAC3sv2 (encoded for example by HDAC3sv2), HDAC3sv3 (encoded for example by HDAC3sv3 or HDAC3sv6), or HDAC3sv4 (encoded for example by HDAC3sv4 or HDAC3sv5).

In particular, HDAC3sv1.1 polynucleotides do not have the polynucleotide regions that comprise exons 3, 4, 5, and 6 of the HDAC3 gene. HDAC3sv1.2 polynucleotides do not have the polynucleotide regions that comprise exons 1, 2, 3, 4, 5, and 6, as well as the first 85 nucleotides of exon 7, of the HDAC3 gene. HDAC3sv2 polynucleotides do not have the polynucleotide regions that comprise exons 3 and 4 of the HDAC3 gene. HDAC3sv3 polynucleotides do not have the polynucleotide region that comprises exon 3 of the HDAC3 gene. HDAC3sv4 polynucleotides have an additional polynucleotide region that comprises intron 4 of the HDAC3 gene. HDAC3sv5 polynucleotides have additional polynucleotide regions that comprise introns 4 and 5 of the HDAC3 gene. HDAC3sv6 polynucleotides do not have the polynucleotide regions that comprise exons 3, 11, and 13 of the HDAC3 gene, and have an additional polynucleotide region that comprises intron 5 of the HDAC3 gene.

Regions in HDAC3sv1.1, HDAC3sv1.2, HDAC3sv2, HDAC3sv3, HDAC3sv4, HDAC3sv5, or HDAC3sv6 nucleic acid that do not encode for HDAC3sv1.1, HDAC3sv1.2, HDAC3sv2, HDAC3sv3, or HDAC3sv4, or are not found in SEQ ID NO 1, SEQ ID NO 3, SEQ ID NO 5, SEQ ID NO 7, SEQ ID NO 9, SEQ ID NO 20, or SEQ ID NO 21, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding HDAC3sv1.1, HDAC3sv1.2, HDAC3sv2, HDAC3sv3, or HDAC3sv4 related proteins from different sources. Obtaining nucleic acids HDAC3sv1.1, HDAC3sv1.2, HDAC3sv2, HDAC3sv3, or HDAC3sv4 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

HDAC3sv1.1, HDAC3sv1.2, HDAC3sv2, HDAC3sv3, HDAC3sv4, HDAC3sv5, or HDAC3sv6 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2nd Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2nd Edition, Cold Spring Harbor Laboratory Press, 1989, and U.S. Pat. No. 5,480,784.

HDAC3sv1.1, HDAC3sv1.2, HDAC3sv2, HDAC3sv3, HDAC3sv4, HDAC3sv5, and HDAC3sv6

Probes

Probes for HDAC3sv1.1, HDAC3sv1.2, HDAC3sv2, HDAC3sv3, HDAC3sv4, HDAC3sv5, or HDAC3sv6 contain a region that can specifically hybridize to HDAC3sv1.1, HDAC3sv1.2, HDAC3sv2, HDAC3sv3, HDAC3sv4, HDAC3sv5, or HDAC3sv6 target nucleic acids, respectively, under appropriate hybridization conditions and can distinguish HDAC3sv1.1, HDAC3sv1.2, HDAC3sv2, HDAC3sv3, HDAC3sv4, HDAC3sv5, or HDAC3sv6 nucleic acids from each other and from non-target nucleic acids. Probes for HDAC3sv1.1, HDAC3sv1.2, HDAC3sv2, HDAC3sv3, HDAC3sv4, HDAC3sv5, or HDAC3sv6 can also contain nucleic acid regions that are not complementary with HDAC3sv1.1, HDAC3sv1.2, HDAC3sv2, HDAC3sv3, HDAC3sv4, HDAC3sv5, or HDAC3sv6 nucleic acids.

In embodiments where, for example, HDAC3sv1.1, HDAC3sv2, HDAC3sv3, HDAC3sv4, HDAC3sv5, or HDAC3sv6 polynucleotide probes are used in hybridization assays to specifically detect the presence of HDAC3sv1.1, HDAC3sv2, HDAC3sv3, HDAC3sv4, HDAC3sv5, or HDAC3sv6 polynucleotides in samples, the HDAC3sv1.1, HDAC3sv2, HDAC3sv3, HDAC3sv4, HDAC3sv5, or HDAC3sv6 polynucleotides comprise at least 20 nucleotides of the HDAC3sv1.1, HDAC3sv2, HDAC3sv3, HDAC3sv4, HDAC3sv5, or HDAC3sv6 sequence that correspond to the respective novel exon junction polynucleotide regions.

In particular, for detection of HDAC3sv1.1, the probe comprises at least 20 nucleotides of the HDAC3sv1.1 sequence that corresponds to an exon junction polynucleotide created by the alternative splicing of exon 2 to exon 7 of the primary transcript of the HDAC3 gene (see FIGS. 1A and 1B). For example, the polynucleotide sequence: 5′ GAAGATGATCGTACCACCCT 3′ [SEQ ID NO 22] represents one embodiment of such an inventive HDAC3sv1.1 polynucleotide wherein a first 10 nucleotides region is complementary and hybridizable to the 3′ end of exon 2 of the HDAC3 gene and a second 10 nucleotide region is complementary and hybridizable to the 5′ end of exon 7 of the HDAC3 gene (see FIG. 1B).

In another embodiment, for detection of HDAC3sv2, the probe comprises at least 20 nucleotides of the HDAC3sv2 sequence that corresponds to an exon junction polynucleotide created by the alternative splicing of exon 2 to exon 5 of the primary transcript of the HDAC3 gene (see FIGS. 1A and 1B). For example, the polynucleotide sequence: 5′ GAAGATGATCATCTGTGATA 3′ [SEQ ID NO 23] represents one embodiment of such an inventive HDAC3sv2 polynucleotide wherein a first 10 nucleotides region is complementary and hybridizable to the 3′ end of exon 2 of the HDAC3 gene and a second 10 nucleotide region is complementary and hybridizable to the 5′ end of exon 5 of the HDAC3 gene (see FIG. 1B).

In another embodiment, for detection of HDAC3sv3 or HDAC3sv6, the probe comprises at least 20 nucleotides of the HDAC3sv3 or HDAC3sv6 sequence that corresponds to an exon junction polynucleotide created by the alternative splicing of exon 2 to exon 4 of the primary transcript of the HDAC3 gene (see FIGS. 1A and 1B). For example, the polynucleotide sequence: 5′ GAAGATGATCCCCAGTGTTT 3′ [SEQ ID NO 24] represents one embodiment of such an inventive HDAC3sv3 or HDAC3sv6 polynucleotide wherein a first 10 nucleotides region is complementary and hybridizable to the 3′ end of exon 2 of the HDAC3 gene and a second 10 nucleotide region is complementary and hybridizable to the 5′ end of exon 4 of the HDAC3 gene (see FIG. 1B).

In another embodiment, for detection of HDAC3sv4 or HDAC3sv5, the probe comprises at least 20 nucleotides of the HDAC3sv4 or HDAC3sv5 sequence that corresponds to an exon junction polynucleotide created by the alternative splicing of exon 4 to intron 4 of the primary transcript of the HDAC3 gene (see FIGS. 1A and 1B). For example, the polynucleotide sequence: 5′ GAACAACAAGGTGACATAGT 3′ [SEQ ID NO 25] represents one embodiment of such an inventive HDAC3sv4 or HDAC3sv5 polynucleotide wherein a first 10 nucleotides region is complementary and hybridizable to the 3′ end of exon 4 of the HDAC3 gene and a second 10 nucleotide region is complementary and hybridizable to the 5′ end of intron 4 of the HDAC3 gene (see FIG. 1B).

In another example, the probe comprises at least 20 nucleotides of the HDAC3sv4 or HDAC3sv5 sequence that corresponds to an exon junction polynucleotide created by the alternative splicing of intron 4 to exon 5 of the primary transcript of the HDAC3 gene (see FIGS. 1A and 1B). For example, the polynucleotide sequence: 5′ TGTCTTTCAGATCT GTGATA 3′ [SEQ ID NO 26] represents one embodiment of such an inventive HDAC3sv4 or HDAC3sv5 polynucleotide wherein a first 10 nucleotides region is complementary and hybridizable to the 3′ end of intron 4 of the HDAC3 gene and a second 10 nucleotide region is complementary and hybridizable to the 5′ end of exon 5 of the HDAC3 gene (see FIG. 1B).

In another embodiment, for detection of HDAC3sv5 or HDAC3sv6, the probe comprises at least 20 nucleotides of the HDAC3sv5 or HDAC3sv6 sequence that corresponds to an exon junction polynucleotide created by the alternative splicing of exon 5 to intron 5 of the primary transcript of the HDAC3 gene (see FIGS. 1A and 1B). For example, the polynucleotide sequence: 5′ GAAGTTTGAGGTGAGTGAGG 3′ [SEQ ID NO 27] represents one embodiment of such an inventive HDAC3sv5 polynucleotide wherein a first 10 nucleotide region is complementary and hybridizable to the 3′ end of exon 5 of the HDAC3 gene and a second 10 nucleotide region is complementary and hybridizable to the 5′ end of intron 5 of the HDAC3 gene (see FIG. 1B).

In another example, the probe comprises at least 20 nucleotides of the HDAC3sv5 or HDAC3sv6 sequence that corresponds to an exon junction polynucleotide created by the alternative splicing of intron 5 to exon 6 of the primary transcript of the HDAC3 gene (see FIGS. 1A and 1B). For example, the polynucleotide sequence: 5′ CTTGCCATAGGCCTCTG GCT 3′ [SEQ ID NO 28] represents one embodiment of such an inventive HDAC3sv5 polynucleotide wherein a first 10 nucleotides region is complementary and hybridizable to the 3′ end of intron 5 of the HDAC3 gene and a second 10 nucleotide region is complementary and hybridizable to the 5′ end of exon 6 of the HDAC3 gene (see FIG. 1B).

In another embodiment, for the detection of HDAC3sv6, the probe comprises at least 20 nucleotides of the HDAC3sv6 sequence that corresponds to an exon junction polynucleotide created by the alternative splicing of exon 10 to exon 12 of the primary transcript of the HDAC3 gene (see FIGS. 1A and 1B). For example, the polynucleotide sequence: 5′ GAGGGCATGGGACATATGAG 3′ [SEQ ID NO 29] represents one embodiment of such an inventive HDAC3sv6 polynucleotide wherein a first 10 nucleotides region is complementary and hybridizable to the 3′ end of exon 10 of the HDAC3 gene and a second 10 nucleotide region is complementary and hybridizable to the 5′ end of exon 12 of the HDAC3 gene (see FIG. 1B).

In another example, the probe comprises at least 20 nucleotides of the HDAC3sv6 sequence that corresponds to an exon junction polynucleotide created by the alternative splicing of exon 12 to exon 14 of the primary transcript of the HDAC3 gene (see FIGS. 1A and 1B). For example, the polynucleotide sequence: 5′ CCCTATAGTGTATCTGGACC 3′ [SEQ ID NO 30] represents one embodiment of such an inventive HDAC3sv6 polynucleotide wherein a first 10 nucleotides region is complementary and hybridizable to the 3′ end of exon 12 of the HDAC3 gene and a second 10 nucleotide region is complementary and hybridizable to the 5′ end of exon 14 of the HDAC3 gene (see FIG. 1B).

In some embodiments, the first 20 nucleotides of a HDAC3sv1.1 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 7. In some embodiments, the first 20 nucleotides of a HDAC3sv2 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 5. In some embodiments, the first 20 nucleotides of a HDAC3sv3 or HDAC3sv6 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 4.

In some embodiments, the first 20 nucleotides of a HDAC3sv4 or HDAC3sv5 probe comprise a first continuous region of 5 to 15 nucleotides that is complementary and hybridizable to the 3′ end of exon 4 and a second continuous region of 5 to 15 nucleotides that is complementary and hybridizable to the 5′ end of intron 4. In another example, the first 20 nucleotides of a HDAC3sv4 or HDAC3sv5 probe comprise a first continuous region of 5 to 15 nucleotides that is complementary and hybridizable to the 3′ end of intron 4 and a second continuous region of 5 to 15 nucleotides that is complementary and hybridizable to the 5′ end of exon 5.

In some embodiments, the first 20 nucleotides of a HDAC3sv5 or HDAC3sv6 probe comprise a first continuous region of 5 to 15 nucleotides that is complementary and hybridizable to the 3′ end of exon 5 and a second continuous region of 5 to 15 nucleotides that is complementary and hybridizable to the 5′ end of intron 5. In another example, the first 20 nucleotides of a HDAC3sv5 or HDAC3sv6 probe comprise a first continuous region of 5 to 15 nucleotides that is complementary and hybridizable to the 3′ end of intron 5 and a second continuous region of 5 to 15 nucleotides that is complementary and hybridizable to the 5′ end of exon 6.

In some embodiments, the first 20 nucleotides of a HDAC3sv6 probe comprise a first continuous region of 5 to 15 nucleotides that is complementary and hybridizable to the 3′ end of exon 10 and a second continuous region of 5 to 15 nucleotides that is complementary and hybridizable to the 5′ end of exon 12. In another example, the first 20 nucleotides of a HDAC3sv6 probe comprise a first continuous region of 5 to 15 nucleotides that is complementary and hybridizable to the 3′ end of exon 12 and a second continuous region of 5 to 15 nucleotides that is complementary and hybridizable to the 5′ end of exon 14.

In other embodiments, the HDAC3sv1.1, HDAC3sv2, HDAC3sv3, HDAC3sv4, HDAC3sv5, or HDAC3sv6 polynucleotide comprises at least 40, 60, 80 or 100 nucleotides of the HDAC3sv1.1, HDAC3sv2, HDAC3sv3, HDAC3sv4, HDAC3sv5, or HDAC3sv6 sequence, respectively, that correspond to a junction polynucleotide region created by the alternative splicing of exon 2 to exon 7 in the case of HDAC3sv1.1; that correspond to a junction polynucleotide region created by the alternative splicing of exon 2 to exon 5 in the case of HDAC3sv2; that correspond to a junction polynucleotide region created by the alternative splicing of exon 2 to exon 4 in the case of HDAC3sv3 or HDAC3sv6; that correspond to a junction polynucleotide region created by the alternative splicing of exon 4 to intron 4, or of intron 4 to exon 5 in the case of HDAC3sv4 or HDAC3sv5; that correspond to a junction polynucleotide region created by the alternative splicing of exon 5 to intron 5, or intron 5 to exon 6 in the case of HDAC3sv5 or HDAC3sv6; or in the case of HDAC3sv6 by the alternative splicing of exon 10 to exon 12 or exon 12 to exon 14 of the primary transcript of the HDAC3 gene.

In embodiments involving HDAC3sv1.1, the HDAC3sv1.1 polynucleotide is selected to comprise a first continuous region of 5 to 15 nucleotides that is complementary and hybridizable to the 3′ end of exon 2 and a second continuous region of 5 to 15 nucleotides that is complementary and hybridizable to the 5′ end of exon 7.

Similarly, in embodiments involving HDAC3sv2, the HDAC3sv2 polynucleotide is selected to comprise a first continuous region of 5 to 15 nucleotides that is complementary and hybridizable to the 3′ end of exon 2 and a second continuous region of 5 to 15 nucleotides that is complementary and hybridizable to the 5′ end of exon 5.

Similarly, in embodiments involving HDAC3sv3 or HDAC3SV6, the HDAC3sv3 or HDAC3sv6 polynucleotide is selected to comprise a first continuous region of 5 to 15 nucleotides that is complementary and hybridizable to the 3′ end of exon 2 and a second continuous region of 5 to 15 nucleotides that is complementary and hybridizable to the 5′ end of exon 4.

Similarly, in embodiments involving HDAC3sv4 or HDAC3sv5, the HDAC3sv4 or HDAC3sv5 polynucleotide is selected to comprise a first continuous region of 5 to 15 nucleotides that is complementary and hybridizable to the 3′ end of exon 4 and a second continuous region of 5 to 15 nucleotides that is complementary and hybridizable to the 5′ end of intron 4. In another example involving HDAC3sv4 or HDAC3sv5, the HDAC3sv4 or HDAC3sv5 polynucleotide is selected to comprise a first continuous region of 5 to 15 nucleotides that is complementary and hybridizable to the 3′ end of intron 4 and a second continuous region of 5 to 15 nucleotides that is complementary and hybridizable to the 5′ end of exon 5.

Similarly, in embodiments involving HDAC3sv5 or HDAC3sv6, the HDAC3sv5 or HDAC3sv6 polynucleotide is selected to comprise a first continuous region of 5 to 15 nucleotides that is complementary and hybridizable to the 3′ end of exon 5 and a second continuous region of 5 to 15 nucleotides that is complementary and hybridizable to the 5′ end of intron 5. In another example involving HDAC3sv5 or HDAC3sv6, the HDAC3sv5 or HDAC3sv6 polynucleotide is selected to comprise a first continuous region of 5 to 15 nucleotides that is complementary and hybridizable to the 3′ end of intron 5 and a second continuous region of 5 to 15 nucleotides that is complementary and hybridizable to the 5′ end of exon 6.

Similarly, in embodiments involving HDAC3sv6, the HDAC3sv6 polynucleotide is selected to comprise a first continuous region of 5 to 15 nucleotides that is complementary and hybridizable to the 3′ end of exon 10 and a second continuous region of 5 to 15 nucleotides that is complementary and hybridizable to the 5′ end of exon 12. In another example involving HDAC3sv6, the HDAC3sv6 polynucleotide is selected comprise a first continuous region of 5 to 15 nucleotides that is complementary and hybridizable to the 3′ end of exon 12 and a second continuous region of 5 to 15 nucleotides that is complementary and hybridizable to the 5′ end of exon 14.

As will be apparent to a person of skill in the art, a large number of different polynucleotide sequences from the region of the exon 2 to exon 7 splice junction; the exon 2 to exon 5 splice junction; the exon 2 to exon 4 splice junction; the exon 4 to intron 4 splice junction and the intron 4 to exon 5 splice junction; the exon 5 to intron 5 splice junction and the intron 5 to exon 6 splice junction; the exon 10 to exon 12 splice junction; and the exon 12 to exon 14 splice junction may be selected which will, under appropriate hybridization conditions, have the capacity to detectably hybridize to HDAC3sv1.1, HDAC3sv2, HDAC3sv3, HDAC3sv4, HDAC3sv5, or HDAC3sv6 polynucleotides, respectively, and yet will hybridize to a much less extent or not at all to HDAC3 isoform polynucleotides wherein exon 2 is not spliced to exon 7; wherein exon 2 is not spliced to exon 5; wherein exon 2 is not spliced to exon 4; wherein exon 4 is not spliced to intron 4 and intron 4 is not splice to exon 5; wherein exon 5 is not spliced to intron 5 and intron 5 is not spliced to exon 6; and wherein exon 10 is not spliced to exon 12 and exon 12 is not spliced to exon 14, respectively.

Preferably, non-complementary nucleic acid that is present has a particular purpose such as being a reporter sequence or being a capture sequence. However, additional nucleic acid need not have a particular purpose as long as the additional nucleic acid does not prevent the HDAC3sv1.1, HDAC3sv2, HDAC3sv3, HDAC3sv4, HDAC3sv5, or HDAC3sv6 nucleic acid from distinguishing between target polynucleotides, e.g., HDAC3sv1.1, HDAC3sv2, HDAC3sv3, HDAC3sv4, HDAC3sv5, or HDAC3sv6 polynucleotides, and non-target polynucleotides, including, but not limited to HDAC3 polynucleotides not comprising the exon 2 to exon 7 splice junction, the exon 2 to exon 5 splice junction, the exon 2 to exon 4 splice junctions, the exon 4 to intron 4 and intron 4 to exon 5 splice junctions, the exon 5 to intron 5 and intron 5 to exon 6 splice junctions, or the exon 10 to exon 12 and exon 12 to exon 14 splice junctions found in HDAC3sv1.1, HDAC3sv2, HDAC3sv3, HDAC3sv4, HDAC3sv5, or HDAC3sv6, respectively.

In embodiments where, for example, HDAC3sv1.2 polynucleotide probes are used in hybridization assays to specifically detect the presence of HDAC3sv1.2 polynucleotides in samples, the HDAC3sv1.2 polynucleotides comprise at least 20 nucleotides of the HDAC3sv1.2 sequence that correspond to the first 20 nucleotides at the amino terminus of the HDAC3sv1.2 polynucleotide. For example, the polynucleotide sequence: 5′ ATGACGGTGTCCTTCCACAA 3′ [SEQ ID NO 31] represents one embodiment of such an inventive HDAC3sv1.2 polynucleotide wherein the 20 nucleotides region is complementary and hybridizable to the 20 nucleotides starting with the “ATG” codon, 86 nucleotides downstream of the 5′ end of exon 7 of the HDAC3 gene.

In other embodiments, the HDAC3sv1.2 polynucleotide comprises at least 40, 60, 80 or 100 nucleotides of the HDAC3sv1.2 sequence that correspond to the first 40, 60, 80 or 100 nucleotides, respectively, starting with the “ATG” codon 86 nucleotides downstream of the 5′ end of exon 7 of the primary transcript of the HDAC3 gene.

Preferably, non-complementary nucleic acid that is present has a particular purpose such as being a reporter sequence or being a capture sequence. However, additional nucleic acid need not have a particular purpose as long as the additional nucleic acid does not prevent the HDAC3sv1.2 nucleic acid from distinguishing between target polynucleotides, e.g., HDAC3sv1.2 polynucleotides, and non-target polynucleotides.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2nd Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

HDAC3sv1.1, HDAC3sv1.2, HDAC3sv2, HDAC3sv3, HDAC3sv4, HDAC3sv5, or HDAC3sv6 polynucleotides, such as those comprising SEQ ID NO 1, SEQ ID NO 3, SEQ ID NO 5, SEQ ID NO 7, SEQ ID NO 9, SEQ ID NO 20 and SEQ ID NO 21, respectively, can be used to make HDAC3sv1.1, HDAC3sv1.2, HDAC3sv2, HDAC3sv3, or HDAC3sv4, polypeptides. In particular, HDAC3sv1.1, HDAC3sv1.2, HDAC3sv2, HDAC3sv3, or HDAC3sv4 polypeptides can be expressed from recombinant nucleic acids in a suitable host or in vitro using a translation system. Recombinantly expressed HDAC3sv1.1, HDAC3sv1.2, HDAC3sv2, HDAC3sv3, or HDAC3sv4 polypeptides can be used, for example, in assays to screen for compounds that bind HDAC3sv1.1, HDAC3sv1.2, HDAC3sv2, HDAC3sv3, or HDAC3sv4, respectively. Alternatively, HDAC3sv1.1, HDAC3sv1.2, HDAC3sv2, HDAC3sv3, or HDAC3sv4 polypeptides can also be used to screen for compounds that bind to one or more HDAC3 isoforms but do not bind to HDAC3sv1.1, HDAC3sv1.2, HDAC3sv2, HDAC3sv3, or HDAC3sv4, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146) and pUCTag (ATCC 37460), and. Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 1, SEQ ID NO 3, SEQ ID NO 5, SEQ ID NO 7, SEQ ID NO 9, SEQ ID NO 20, or SEQ ID NO 21 to take into account codon usage of the host. Codon usages of different organisms are well known in the art (see, Ausubel, Current Protocols in Molecular Biology, John Wiley, 1987-1998, Supplement 33 Appendix IC).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

HDAC3sv1.1, HDAC3sv1.2, HDAC3sv2, HDAC3sv3, and HDAC3sv4 Polypeptides

HDAC3sv1.1 polypeptides contain an amino acid sequence comprising, consisting or consisting essentially of SEQ ID NO 2. HDAC3sv1.2 polypeptides contain an amino acid sequence comprising, consisting or consisting essentially of SEQ ID NO 4. HDAC3sv2 polypeptides contain an amino acid sequence comprising, consisting or consisting essentially of SEQ ID NO 6. HDAC3sv3 polypeptides contain an amino acid sequence comprising, consisting or consisting essentially of SEQ ID NO 8. HDAC3sv4 polypeptides contain an amino acid sequence comprising, consisting or consisting essentially of SEQ ID NO 10. HDAC3sv1.1, HDAC3sv1.2, HDAC3sv2, HDAC3sv3, or HDAC3sv4 polypeptides have a variety of uses, such as providing a marker for the presence of HDAC3sv1.1, HDAC3sv1.2, HDAC3sv2, HDAC3sv3, or HDAC3sv4, respectively; use as an immunogen to produce antibodies binding to HDAC3sv1.1, HDAC3sv1.2, HDAC3sv2, HDAC3sv3, or HDAC3sv4, respectively; use as a target to identify compounds binding selectively to HDAC3sv1.1, HDAC3sv1.2, HDAC3sv2, HDAC3sv3, or HDAC3sv4, respectively; or use in an assay to identify compounds that bind to one or more isoforms of HDAC3 but do not bind to or interact with HDAC3sv1.1, HDAC3sv1.2, HDAC3sv2, HDAC3sv3, or HDAC3sv4, respectively.

In chimeric polypeptides containing one or more regions from HDAC3sv1.1, HDAC3sv1.2, HDAC3sv2, HDAC3sv3, or HDAC3sv4 and one or more regions not from HDAC3sv1.1, HDAC3sv1.2, HDAC3sv2, HDAC3sv3, or HDAC3sv4, respectively, the region(s) not from HDAC3sv1.1, HDAC3sv1.2, HDAC3sv2, HDAC3sv3, or HDAC3sv4, respectively, can be used, for example, to achieve a particular purpose or to produce a polypeptide that can substitute for HDAC3sv1.1, HDAC3sv1.2, HDAC3sv2, HDAC3sv3, or HDAC3sv4, or fragments thereof. Particular purposes that can be achieved using chimeric HDAC3sv1.1, HDAC3sv1.2, HDAC3sv2, HDAC3sv3, or HDAC3sv4 polypeptides include providing a marker for HDAC3sv1.1, HDAC3sv1.2, HDAC3sv2, HDAC3sv3, or HDAC3sv4 activity, respectively, enhancing an immune response, and modulating transcription activity or levels of histone deacetylation.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art.

Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional HDAC3sv1.1, HDAC3sv1.2, HDAC3sv2, HDAC3sv3, and HDAC3sv4

Functional HDAC3sv1.1, HDAC3sv1.2, HDAC3sv2, HDAC3sv3, or HDAC3sv4 are different protein isoforms of HDAC3. The identification of the amino acid and nucleic acid sequences of HDAC3sv1.1, HDAC3sv1.2, HDAC3sv2, HDAC3sv3, or HDAC3sv4 provide tools for obtaining functional proteins related to HDAC3sv1.1, HDAC3sv1.2, HDAC3sv2, HDAC3sv3, or HDAC3sv4, respectively, from other sources; for producing HDAC3sv1.1, HDAC3sv1.2, HDAC3sv2, HDAC3sv3, or HDAC3sv4 chimeric proteins; and for producing functional derivatives of SEQ ID NO 2, SEQ ID NO 4, SEQ ID NO 6, SEQ ID NO 8, or SEQ ID NO 10.

HDAC3sv1.1, HDAC3sv1.2, HDAC3sv2, HDAC3sv3, or HDAC3sv4 polypeptides can be readily identified and obtained based on their sequence similarity to HDAC3sv1.1 (SEQ ID NO 2), HDAC3sv1.2 (SEQ ID NO 4), HDAC3sv2 (SEQ ID NO 6), HDAC3sv3 (SEQ ID NO 8), or HDAC3sv4 (SEQ ID NO 10), respectively. In particular, HDAC3sv1.1 lacks the amino acids encoded by exons 3, 4, 5, and 6 of the HDAC3 gene. The deletion of exons 3-6 and the splicing of exon 2 to exon 7 of the HDAC3 hnRNA transcript results in a shift of the protein reading frame at the exon 2 to exon 7 splice junction thereby creating a carboxy-terminal peptide region that is unique to the HDAC3sv1.1 polypeptide as compared to other known HDAC3 isoforms. The frame shift creates a premature termination codon twenty-five nucleotides downstream of the exon 2/exon 7 splice junction. Thus, the HDAC3sv1.1 polypeptide is lacking the amino acids encoded by the nucleotides downstream of the premature stop codon.

The HDAC3sv1.2 carboxy terminal polypeptide lacks the amino acids encoded by the first 561 nucleotides of the HDAC3 gene. Initiation at a downstream AUG of a bicistronic RNA is a fairly common event and can be associated with disease (Meijer and Thomas, 2002 Biochem. J., 367:1-11; Kozak, 2002, Mammalian Genome 13:401-410).

The HDAC3sv2 polypeptides lack the amino acids encoded by exons 3 and 4 of the HDAC3 gene.

The HDAC3sv3 polypeptide lacks the amino acids encoded by exon 3 of the HDAC3 gene. The deletion of exon 3 results in a frame shift, thereby creating a peptide region having amino acids that are unique to the HDAC3sv3 polypeptides. The frame shift creates a premature termination codon twenty-two nucleotides downstream of the exon 2/exon 4 splice junction. Thus, the HDAC3sv3 polypeptide lacks the amino acids encoded by the nucleotides downstream of the premature stop codon.

The HDAC3sv4 polypeptide contains two additional amino acids encoded by the retained intron 4 sequence. Seven nucleotides downstream of the exon 4/intron 4 splice junction there is an in-frame stop codon. Thus, the HDAC3sv4 polypeptide lacks the amino acids encoded by the nucleotides downstream of the intron 4 stop codon.

Both the amino acid and nucleic acid sequences of HDAC3sv1.1, HDAC3sv1.2, HDAC3sv2, HDAC3sv3, or HDAC3sv4 can be used to help identify and obtain HDAC3sv1.1, HDAC3sv1.2, HDAC3sv2, HDAC3sv3, or HDAC3sv4 polypeptides, respectively. For example, SEQ ID NO 1 can be used to produce degenerative nucleic acid probes or primers for identifying and cloning nucleic acid polynucleotides encoding for a HDAC3sv1.1 polypeptide. In addition, polynucleotides comprising, consisting, or consisting essentially of SEQ ID NO 1 or fragments thereof, can be used under conditions of moderate stringency to identify and clone nucleic acids encoding HDAC3sv1.1 polypeptides from a variety of different organisms. The same methods can also be performed with polynucleotides comprising, consisting, or consisting essentially of SEQ ID NO 3, SEQ ID NO 5, SEQ ID NO 7, or SEQ ID NO 9, or fragments thereof, to identify and clone nucleic acids encoding HDAC3sv1.2, HDAC3sv2, HDAC3sv3, and HDAC3sv4, respectively.

The use of degenerative probes and moderate stringency conditions for cloning is well known in the art. Examples of such techniques are described by Ausubel (Current Protocols in Molecular Biology, John Wiley, 1987-1998), and Sambrook, et al., (Molecular Cloning, A Laboratory Manual, 2^{nd} Edition, Cold Spring Harbor Laboratory Press, 1989).

Starting with HDAC3sv1.1, HDAC3sv1.2, HDAC3sv2, HDAC3sv3, or HDAC3sv4 obtained from a particular source, derivatives can be produced. Such derivatives include polypeptides with amino acid substitutions, additions and deletions. Changes to HDAC3sv1.1, HDAC3sv1.2, HDAC3sv2, HDAC3sv3, or HDAC3sv4 to produce a derivative having essentially the same properties should be made in a manner not altering the tertiary structure of HDAC3sv1.1, HDAC3sv1.2, HDAC3sv2, HDAC3sv3, or HDAC3sv4, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

HDAC3sv1.1, HDAC3sv1.2, HDAC3sv2, HDAC3sv3, and HDAC3sv4 Antibodies

Antibodies recognizing HDAC3sv1.1, HDAC3sv1.2, HDAC3sv2, HDAC3sv3, or HDAC3sv4 can be produced using a polypeptide containing SEQ ID NO 2 in the case of HDAC3sv1.1, SEQ ID NO 4 in the case of HDAC3sv1.2, SEQ ID NO 6 in the case of HDAC3sv2, SEQ ID NO 8 in the case of HDAC3sv3, or SEQ ID NO 10 in the case of HDAC3sv4, respectively, or a fragment thereof, as an immunogen. Preferably, a HDAC3sv1.1 polypeptide used as an immunogen consists of a polypeptide of SEQ ID NO 2 or a SEQ ID NO 2 fragment having at least 10 contiguous amino acids in length corresponding to the polynucleotide region representing the junction resulting from the splicing of exon 2 to exon 7 of the HDAC3 gene. Preferably, a HDAC3sv1.2 polypeptide used as an immunogen consists of a polypeptide of SEQ ID NO 4 or a SEQ ID NO 4 fragment having at least 10 contiguous amino acids in length corresponding to amino acids, including and downstream of, the amino terminal methionine of HDAC3sv1.2. Preferably, a HDAC3sv2 polypeptide used as an immunogen consists of a polypeptide derived from SEQ ID NO 6 or a SEQ ID NO 6 fragment, having at least 10 contiguous amino acids in length corresponding to a polynucleotide region representing the junction resulting from the splicing of exon 2 to exon 5 of the HDAC3 gene. Preferably, a HDAC3sv3 polypeptide used as an immunogen consists of a polypeptide derived from SEQ ID NO 8 or a SEQ ID NO 8 fragment, having at least 10 contiguous amino acids in length corresponding to a polynucleotide region representing the junction resulting from the splicing of exon 2 to exon 4 of the HDAC3 gene. Preferably, a HDAC3sv4 polypeptide used as an immunogen consists of a polypeptide derived from SEQ ID NO 10 or a SEQ ID NO 10 fragment having at least 10 contiguous amino acids in length corresponding to a polynucleotide region representing the junction resulting from the splicing of exon 4 to intron 4 of the HDAC3 gene.

In some embodiments where, for example, HDAC3sv1.1 polypeptides are used to develop antibodies that bind specifically to HDAC3sv1.1 and not to other isoforms of HDAC3, the HDAC3sv1.1 polypeptides comprise at least 10 amino acids of the HDAC3sv1.1 polypeptide sequence corresponding to a junction polynucleotide region created by the alternative splicing of exon 2 to exon 7 of the primary transcript the HDAC3 gene (see FIG. 1). For example, the amino acid sequence: amino terminus-YKKMIVPPSG-carboxy terminus [SEQ ID NO 32] represents one embodiment of such an inventive HDAC3sv1.1 polypeptide wherein a first 5 amino acid region is encoded by nucleotide sequence at the 3′ end of exon 2 of the HDAC3 gene and a second 5 amino acid region is encoded by the nucleotide sequence directly after the novel splice junction. Preferably, at least 10 amino acids of the HDAC3sv1.1 polypeptide comprises a first continuous region of 2 to 8 amino acids that is encoded by nucleotides at the 3′ end of exon 2 and a second continuous region of 2 to 8 amino acids that is encoded by nucleotides at the 5′ end of exon 7.

In other embodiments where, for example, HDAC3sv1.2 polypeptides are used to develop antibodies that bind specifically to HDAC3sv1.2 and not to other isoforms of HDAC3, the HDAC3sv1.2 polypeptides comprise at least 10 amino acids at the amino terminus of the HDAC3sv1.2 polypeptide sequence having at least 10 contiguous amino acids in length corresponding to amino acids, including and downstream of, the amino terminal methionine of HDAC3sv1.2. For example, the amino acid sequence: amino terminus-MTVSFHKYGN-carboxy terminus [SEQ ID NO 33], represents one embodiment of such an inventive HDAC3sv1.2 polypeptide wherein a first 10 amino acid region is encoded by a nucleotide sequence starting with the “ATG” codon 86 nucleotides downstream of the 5′ end of exon 7 of the HDAC3 gene.

In other embodiments where, for example, HDAC3sv2 polypeptides are used to develop antibodies that bind specifically to HDAC3sv2 and not to other HDAC3 isoforms, the HDAC3sv2 polypeptides comprise at least 10 amino acids of the HDAC3sv2 polypeptide sequence corresponding to a junction polynucleotide region created by the alternative splicing of exon 2 to exon 5 of the primary transcript of the HDAC3 gene (see FIG. 1). For example, the amino acid sequence: amino terminus-YKKMIICDIA-carboxy terminus [SEQ ID NO 34], represents one embodiment of such an inventive HDAC3sv2 polypeptide wherein a first 5 amino acid region is encoded by a nucleotide sequence at the 3′ end of exon 2 of the HDAC3 gene and a second 5 amino acid region is encoded by a nucleotide sequence directly after the novel splice junction. Preferably, at least 10 amino acids of the HDAC3sv2 polypeptide comprises a first continuous region of 2 to 8 amino acids that is encoded by nucleotides at the 3′ end of exon 2 and a second continuous region of 2 to 8 amino acids that is encoded by nucleotides at the 5′ end exon 5.

In other embodiments where, for example, HDAC3sv3 polypeptides are used to develop antibodies that bind specifically to HDAC3sv3 and not to other HDAC3 isoforms, the HDAC3sv3 polypeptides comprise at least 10 amino acids of the HDAC3sv3 polypeptide sequence corresponding to a junction polynucleotide region created by the alternative splicing of exon 2 to exon 4 of the primary transcript of the HDAC3 gene (see FIG. 1). For example, the amino acid sequence: amino terminus-YKKMIPSVSR-carboxy terminus [SEQ ID NO 35], represents one embodiment of such an inventive HDAC3sv3 wherein a first 5 amino acid region is encoded by a nucleotide sequence at the 3′ end of exon 2 of the HDAC3 gene and a second 5 amino acid region is encoded by a nucleotide sequence directly after the novel splice junction. Preferably, at least 10 amino acids of the HDAC3sv3 polypeptides comprises a first continuous region of 3 to 8 amino acids that is encoded by nucleotides at the 3′ end of exon 2 and a second continuous region of 2 to 7 amino acids that is encoded by nucleotides at the 5′ end exon 4.

In other embodiments where, for example, HDAC3sv4 polypeptides are used to develop antibodies that bind specifically to HDAC3sv4 and not to other HDAC3 isoforms, the HDAC3sv4 polypeptides comprise at least 10 amino acids of the HDAC3sv4 polypeptide sequence corresponding to a junction polynucleotide region created by the alternative splicing of exon 4 to intron 4 of the primary transcript of the HDAC3 gene (see FIG. 1). For example, the amino acid sequence: amino terminus-GATQLNNKVT-carboxy terminus [SEQ ID NO 36], represents one embodiment of such an inventive HDAC3sv4 polypeptide wherein a first 8 amino acid region is encoded by a nucleotide sequence at the 3′ end of exon 4 of the HDAC3 gene and a second 2 amino acid region is encoded by a nucleotide sequence directly after the novel splice junction.

In other embodiments, HDAC3sv1.1-specific antibodies are made using an HDAC3sv1.1 polypeptide that comprises at least 20, 30, 40 or 50 amino acids of the HDAC3sv1.1 sequence that corresponds to a junction polynucleotide region created by the alternative splicing of exon 2 to exon 7 of the primary transcript of the HDAC3 gene. In each case the HDAC3sv1.1 polypeptides are selected to comprise a first continuous region of at least 5 to 15 amino acids that is encoded by nucleotides at the 3′ end of exon 2 and a second continuous region of 5 to 15 amino acids that is encoded by nucleotides directly after the novel splice junction.

In other embodiments, HDAC3sv1.2-specific antibodies are made using an HDAC3sv1.2 polypeptide that comprises at least 20, 30, 40, or 50 amino acids of the HDAC3sv1.2 sequence that corresponds to a polynucleotide region encoding amino acids, including and downstream of, the methionine codon located 86 nucleotides downstream of the 5′ end of exon 7 of the primary transcript of the HDAC3 gene.

In other embodiments, HDAC3sv2-specific antibodies are made using an HDAC3sv2 polypeptide that comprises at least 20, 30, 40 or 50 amino acids of the HDAC3sv2 sequence that corresponds to a junction polynucleotide region created by the alternative splicing of exon 2 to exon 5 of the primary transcript of the HDAC3 gene. In each case the HDAC3sv2 polypeptides are selected to comprise a first continuous region of at least 5 to 15 amino acids that is encoded by nucleotides at the 3′ end of exon 2 and a second continuous region of 5 to 15 amino acids that is encoded by nucleotides directly after the novel splice junction.

In other embodiments, HDAC3sv3-specific antibodies are made using an HDAC3sv3 polypeptide that comprises at least 20, 30, 40 or 50 amino acids of the HDAC3sv3 sequence that corresponds to a junction polynucleotide region created by the alternative splicing of exon 2 to exon 4 of the primary transcript of the HDAC3 gene. In each case the HDAC3sv3 polypeptides are selected to comprise a first continuous region of at least 13 to 15 amino acids that is encoded by nucleotides at the 3′ end of exon 2 and a second continuous region of 5 to 7 amino acids that is encoded by nucleotides directly after the novel splice junction.

In other embodiments, HDAC3sv4-specific antibodies are made using an HDAC3sv4 polypeptide that comprises at least 20, 30, 40 or 50 amino acids of the HDAC3sv4 sequence that corresponds to a junction polynucleotide region created by the alternative splicing of exon 4 to intron 4 of the primary transcript of the HDAC3 gene. In each case the HDAC3sv4 polypeptides are selected to comprise a first continuous region of at least 18 amino acids that is encoded by nucleotides at the 3′ end of exon 4 and a second continuous region of 2 amino acids that is encoded by nucleotides directly after the novel splice junction.

Antibodies to HDAC3sv1.1, HDAC3sv1.2, HDAC3sv2, HDAC3sv3, or HDAC3sv4 have different uses, such as to identify the presence of HDAC3sv1.1, HDAC3sv1.2, HDAC3sv2, HDAC3sv3, or HDAC3sv4, respectively, and to isolate HDAC3sv1.1, HDAC3sv1.2, HDAC3sv2, HDAC3sv3, or HDAC3sv4 polypeptides, respectively. Identifying the presence of HDAC3sv1.1 can be used, for example, to identify cells producing HDAC3sv1.1. Such identification provides an additional source of HDAC3sv1.1 and can be used to distinguish cells known to produce HDAC3sv1.1 from cells that do not produce HDAC3sv1.1. For example, antibodies to HDAC3sv1.1 can distinguish human cells expressing HDAC3sv1.1 from human cells not expressing HDAC3sv1.1 or non-human cells (including bacteria) that do not express HDAC3sv1.1. Such HDAC3sv1.1 antibodies can also be used to determine the effectiveness of HDAC3sv1.1 ligands, using techniques well known in the art, to detect and quantify changes in the protein levels of HDAC3sv1.1 in cellular extracts, and in situ immunostaining of cells and tissues. In addition, the same above-described utilities also exist for HDAC3sv1.2-specific antibodies, HDAC3sv2-specific antibodies, HDAC3sv3-specific antibodies and HDAC3sv4-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

HDAC3sv1.1, HDAC3sv1.2, HDAC3sv2, HDAC3sv3, and HDAC3sv4 Binding Assay

A number of compounds known to modulate histone deacetylase activity have been disclosed (see for example, U.S. patent application ser. No. 20020061860). Methods for screening these compounds for their effects on histone deacetylase activity have also been disclosed (see for example, Kramer et al., 2001 Trends in Endocrinology & Metabolism 12, 294-300). Some organic compounds that may block histone deacetylase activity have been claimed to be potentially useful treating acute myeloid leukemia (Kramer et al., 2001 Trends in Endocrinology & Metabolism 12, 294-300). A person skilled in the art may use these methods to screen HDAC3sv1.1, HDAC3sv1.2, HDAC3sv2, HDAC3sv3, or HDAC3sv4 polypeptides for compounds that bind to, and in some cases functionally alter, each respective histone deacetylase isoform protein.

HDAC3sv1.1, HDAC3sv1.2, HDAC3sv2, HDAC3sv3, HDAC3sv4, or fragments thereof, can be used in binding studies to identify compounds binding to or interacting with HDAC3sv1.1, HDAC3sv1.2, HDAC3sv2, HDAC3sv3, HDAC3sv4, or fragments thereof, respectively. In one embodiment, the HDAC3sv1.1, or a fragment thereof, can be used in binding studies with a different HDAC3 isoform protein, or a fragment thereof, to identify compounds that: bind to or interact with HDAC3sv1.1 and other HDAC3 isoforms; or bind to or interact with one or more other HDAC3 isoforms and not with HDAC3sv1.1. Alternatively, similar “counter-screening” binding studies can be performed to identify compounds that bind to one or more different HDAC3 isoforms but do not bind to one or more different isoforms of a different HDAC protein, such as, for example, HDAC6, HDAC7A or HDAC9. A similar series of compound activity screens can, of course, also be performed using HDAC3sv1.2, HDAC3sv2, HDAC3sv3, or HDAC3sv4 rather than, or in addition to, HDAC3sv1.1. Such binding studies can be performed using different formats including competitive and non-competitive formats. Further competition studies can be carried out using additional compounds determined to bind to HDAC3sv1.1, HDAC3sv1.2, HDAC3sv2, HDAC3sv3, HDAC3sv4 or other HDAC3 isoforms.

The particular HDAC3sv1.1, HDAC3sv1.2, HDAC3sv2, HDAC3sv3, or HDAC3sv4 amino acid sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using HDAC3sv1.1 expressed from a recombinant nucleic acid. Alternatively, recombinantly expressed HDAC3sv1.1 consists of the SEQ ID NO 2 amino acid sequence. In addition, binding studies are performed using HDAC3sv1.2 expressed from a recombinant nucleic acid. Alternatively, recombinantly expressed HDAC3sv1.2 consists of the SEQ ID NO 4 amino acid sequence. In addition, binding studies are performed using HDAC3sv2 expressed from a recombinant nucleic acid. Alternatively, recombinantly expressed HDAC3sv2 consists of the SEQ ID NO 6 amino acid sequence. In addition, binding studies are performed using HDAC3sv3 expressed from a recombinant nucleic acid. Alternatively, recombinantly expressed HDAC3sv3 consists of the SEQ ID NO 8 amino acid sequence. In addition, binding studies are performed using HDAC3sv4 expressed from a recombinant nucleic acid. Alternatively, recombinantly expressed HDAC3sv4 consists of the SEQ ID NO 10 amino acid sequence.

Binding assays can be performed using individual compounds or preparations containing different numbers of compounds. A preparation containing different numbers of compounds having the ability to bind to HDAC3sv1.1, HDAC3sv1.2, HDAC3sv2, HDAC3sv3, or HDAC3sv4 can be divided into smaller groups of compounds that can be tested to identify the compound(s) binding to HDAC3sv1.1, HDAC3sv1.2, HDAC3sv2, HDAC3sv3, or HDAC3sv4, respectively.

Binding assays can be performed using recombinantly produced HDAC3sv1.1, HDAC3sv1.2, HDAC3sv2, HDAC3sv3, or HDAC3sv4 present in different environments. Such environments include, for example, cell extracts and purified cell extracts containing a HDAC3sv1.1, HDAC3sv1.2, HDAC3sv2, HDAC3sv3, or HDAC3sv4 recombinant nucleic acid; and also include, for example, the use of a purified HDAC3sv1.1, HDAC3sv1.2, HDAC3sv2, HDAC3sv3, or HDAC3sv4 polypeptide produced by recombinant means which is introduced into different environments.

In one embodiment of the invention, a binding method is provided for screening for a compound able to bind selectively to HDAC3sv1.1. The method comprises the steps: providing a HDAC3sv1.1 polypeptide comprising SEQ ID NO 2; providing a HDAC3 isoform polypeptide that is not HDAC3sv1.1; contacting the HDAC3sv1.1 polypeptide and the HDAC3 isoform polypeptide that is not HDAC3sv1.1 with a test preparation comprising one or more test compounds; and then determining the binding of the test preparation to the HDAC3sv1.1 polypeptide and to the HDAC3 isoform polypeptide that is not HDAC3sv1.1, wherein a test preparation that binds to the HDAC3sv1.1 polypeptide, but does not bind to HDAC3 isoform polypeptide that is not HDAC3sv1.1, contains one or more compounds that selectively binds to HDAC3sv1.1.

In one embodiment of the invention, a binding method is provided for screening for a compound able to bind selectively to HDAC3sv1.2. The method comprises the steps: providing a HDAC3sv1.2 polypeptide comprising SEQ ID NO 4; providing a HDAC3 isoform polypeptide that is not HDAC3sv1.2; contacting the HDAC3sv1.2 polypeptide and the HDAC3 isoform polypeptide that is not HDAC3sv1.2 with a test preparation comprising one or more test compounds; and then determining the binding of the test preparation to the HDAC3sv1.2 polypeptide and to the HDAC3 isoform polypeptide that is not HDAC3sv1.2, wherein a test preparation that binds to the HDAC3sv1.2 polypeptide, but does not bind to HDAC3 isoform polypeptide that is not HDAC3sv1.2, contains one or more compounds that selectively binds to HDAC3sv1.2.

In another embodiment of the invention, a binding method is provided for screening for a compound able to bind selectively to HDAC3sv2. The method comprises the steps: providing a HDAC3sv2 polypeptide comprising SEQ ID NO 6; providing a HDAC3 isoform polypeptide that is not HDAC3sv2; contacting the HDAC3sv2 polypeptide and the HDAC3 isoform polypeptide that is not HDAC3sv2 with a test preparation comprising one or more test compounds; and then determining the binding of the test preparation to the HDAC3sv2 polypeptide and to the HDAC3 isoform polypeptide that is not HDAC3sv2, wherein a test preparation that binds to the HDAC3sv2 polypeptide, but does not bind to HDAC3 isoform polypeptide that is not HDAC3sv2, contains one or more compounds that selectively binds to HDAC3sv2.

In another embodiment of the invention, a binding method is provided for screening for a compound able to bind selectively to HDAC3sv3. The method comprises the steps: providing a HDAC3sv3 polypeptide comprising SEQ ID NO 8; providing a HDAC3 isoform polypeptide that is not HDAC3sv3; contacting the HDAC3sv3 polypeptide and the HDAC3 isoform polypeptide that is not HDAC3sv3 with a test preparation comprising one or more test compounds; and then determining the binding of the test preparation to the HDAC3sv3 polypeptide and to the HDAC3 isoform polypeptide that is not HDAC3sv3, wherein a test preparation that binds to the HDAC3sv3 polypeptide, but does not bind to HDAC3 isoform polypeptide that is not HDAC3sv3, contains one or more compounds that selectively binds to HDAC3sv3.

In another embodiment of the invention, a binding method is provided for screening for a compound able to bind selectively to HDAC3sv4. The method comprises the steps: providing a HDAC3sv4 polypeptide comprising SEQ ID NO 10; providing a HDAC3 isoform polypeptide that is not HDAC3sv4; contacting the HDAC3sv4 polypeptide and the HDAC3 isoform polypeptide that is not HDAC3sv4 with a test preparation comprising one or more test compounds; and then determining the binding of the test preparation to the HDAC3sv4 polypeptide and to the HDAC3 isoform polypeptide that is not HDAC3sv4, wherein a test preparation that binds to the HDAC3sv4 polypeptide, but does not bind to HDAC3 isoform polypeptide that is not HDAC3sv4, contains one or more compounds that selectively binds to HDAC3sv4.

In another embodiment of the invention, a binding method is provided for screening for a compound able to bind selectively to a HDAC3 isoform polypeptide that is not HDAC3sv1.1. The method comprises the steps: providing a HDAC3sv1.1 polypeptide comprising SEQ ID NO 2; providing a HDAC3 isoform polypeptide that is not HDAC3sv1.1; contacting the HDAC3sv1.1 polypeptide and the HDAC3 isoform polypeptide that is not HDAC3sv1.1 with a test preparation comprising one or more test compounds; and then determining the binding of the test preparation to the HDAC3sv1.1 polypeptide and the HDAC3 isoform polypeptide that is not HDAC3sv1.1, wherein a test preparation that binds the HDAC3 isoform polypeptide that is not HDAC3sv1.1,but does not bind the HDAC3sv1.1, contains a compound that selectively binds the HDAC3 isoform polypeptide that is not HDAC3sv1.1.

Alternatively, the above method can be used to identify compounds that bind selectively to a HDAC3 isoform polypeptide that is not HDAC3sv1.2 by performing the method with HDAC3sv1.2 protein comprising SEQ ID NO 4. Alternatively, the above method can be used to identify compounds that bind selectively to a HDAC3 isoform polypeptide that is not HDAC3sv2 by performing the method with HDAC3sv2 protein comprising SEQ ID NO 6. Alternatively, the above method can be used to identify compounds that bind selectively to a HDAC3 isoform polypeptide that is not HDAC3sv3 by performing the method with HDAC3sv3 protein comprising SEQ ID NO 8. Alternatively, the above method can be used to identify compounds that bind selectively to a HDAC3 isoform polypeptide that is not HDAC3sv4 by performing the method with HDAC3sv4 protein comprising SEQ ID NO 10.

The above-described selective binding assays can also be performed with a polypeptide fragment of HDAC3sv1.1, HDAC3sv2, HDAC3sv3, or HDAC3sv4, wherein the polypeptide fragment comprises at least 10 consecutive amino acids that are coded by a nucleotide sequence that bridges the junction created by the splicing of the 3′ end of exon 2 to the 5′ end of exon 7 in the case of HDAC3sv1.1; by a nucleotide sequence that bridges the junction created by the splicing of the 3′ end of exon 2 to the 5′ end of exon 5 in the case of HDAC3sv2; by a nucleotide sequence that bridges the junction created by the splicing of the 3′ end of exon 2 to the 5′ end of exon 4 in the case of HDAC3sv3; or by a nucleotide sequence that bridges the junction created by the splicing of the 3′ end of exon 4 to the 5′ end of intron 4 in the case of HDAC3sv4. Similarly, the selective binding assays may also be performed using a polypeptide fragment of an DAC3 isoform polypeptide that is not DAC3sv1.1, HDAC3sv2, HDAC3sv3, or HDAC3sv4, wherein the polypeptide fragment comprises at least 10 consecutive amino acids that are coded by: a) a nucleotide sequence that is contained within exon 3, 4, 5, or 6, of the HDAC3 gene; or b) a nucleotide sequence that bridges the junction created by the splicing of the 3′ end of exon 2 to the 5′ end of exon 3, the splicing of the 3′ end of exon 3 to the 5′ end of exon 4, the splicing of the 3′ end of exon 4 to the 5′ end of exon 5, the splicing of the 3′ end of exon 5 to the 5′ end of exon 6, or the splicing of the 3′ end of exon 6 to the 5′ end of exon 7 of the HDAC3 gene.

Histone Deacetylase HDAC3 Functional Assays

The identification of HDAC3sv1.1, HDAC3sv1.2, HDAC3sv2, HDAC3sv3, and HDAC3sv4 as splice variants of HDAC3 provides a means for screening for compounds that bind to HDAC3sv1.1, HDAC3sv1.2, HDAC3sv2, HDAC3sv3, and/or HDAC3sv4 protein thereby altering the ability of the HDAC3sv1.1, HDAC3sv1.2, HDAC3sv2, HDAC3sv3, and/or HDAC3sv4 polypeptide to bind to Trichostatin A or any other inhibitor compound, or to perform enzymatic assay for histone deacetylase, including any HDAC3 sub-reactions as described, for example by Yang et al., J. Biol. Chem. 272, 28001-28007; Emiliani, S., 1998, Proc. Natl. Acad. Sci. U.S.A. 95, 2795-2800).

Assays involving a functional HDAC3sv1.1, HDAC3sv1.2, HDAC3sv2, HDAC3sv3, or HDAC3sv4 polypeptide can be employed for different purposes, such as selecting for compounds active at HDAC3sv1.1, HDAC3sv1.2, HDAC3sv2, HDAC3sv3, or HDAC3sv4; evaluating the ability of a compound to effect histone deacetylase activity of each respective splice variant polypeptide; and mapping the activity of different HDAC3sv1.1, HDAC3sv1.2, HDAC3sv2, HDAC3sv3, and HDAC3sv4 regions. HDAC3sv1.1, HDAC3sv1.2, HDAC3sv2, HDAC3sv3, and HDAC3sv4 activity can be measured using different techniques such as: detecting a change in the intracellular conformation of HDAC3sv1.1, HDAC3sv1.2, HDAC3sv2, HDAC3sv3, or HDAC3sv4; detecting a change in the intracellular location of HDAC3sv1.1, HDAC3sv1.2, HDAC3sv2, HDAC3sv3, or HDAC3sv4; detecting the amount of binding of Trichostatin A compound to HDAC3sv1.1, HDAC3sv1.2, HDAC3sv2, HDAC3sv3, or HDAC3sv4; or measuring the level of histone deacetylation activity of HDAC3sv1.1, HDAC3sv1.2, HDAC3sv2, HDAC3sv3, or HDAC3sv4.

Recombinantly expressed HDAC3sv1.1, HDAC3sv1.2, HDAC3sv2, HDAC3sv3, and HDAC3sv4 can be used to facilitate determining whether a compound is active at HDAC3sv1.1, HDAC3sv1.2, DAC3sv2, HDAC3sv3, and HDAC3sv4. For example, HDAC3sv1.1, HDAC3sv1.2, HDAC3sv2, HDAC3sv3, and HDAC3sv4 can be expressed by an expression vector in a cell line and used in a co-culture growth assay, such as described in WO 99/59037, to identify compounds that bind to HDAC3sv1.1, HDAC3sv1.2, HDAC3sv2, HDAC3sv3, and HDAC3sv4. For example, HDAC3sv1.1 can be expressed by an expression vector in a human kidney cell line 293 and used in a co-culture growth assay, such as described in U.S. patent application ser. no 20020061860, to identify compounds that bind to HDAC3sv1.1. A similar strategy can be used for HDAC3sv1.2, HDAC3sv2, HDAC3sv3, or HDAC3sv4.

Techniques for measuring histone deacetylase activity are well known in the art (Hendzel et al., 1991, J. Biol. Chem. 266, 21936-21942; Yang et al., 1997, J. Biol. Chem. 272, 28001-28007). In particular, Emliani et al. (1998, Proc. Natl. Acad. Sci. 95, 2795-2800) report methods for expressing a recombinant fragment of the HDAC3 gene tagged with glutathione S-transferase-epitope under the control of T7 RNA polymerase promoter in E. coli to produce truncated HDAC3 polypeptides comprising the catalytic domain of HDAC3. Yang et al. (1997, J. Biol. Chem. 272, 28001-28007) also describe methods for in vitro transcription-translation coupled assays for immunopurifying the expressed epitope-tagged HDAC3 polypeptides from E. coli extracts for use in histone deacetylase enzyme assay. Large varieties of other assays have been used to investigate the properties of HDAC3 and therefore would also be applicable to the measurement of HDAC3sv1.1, HDAC3sv1.2, HDAC3sv2, HDAC3sv3, or HDAC3sv4 functions.

HDAC3sv1.1, HDAC3sv1.2, HDAC3sv2, HDAC3sv3, or HDAC3sv4 functional assays can be performed using cells expressing HDAC3sv1.1, HDAC3sv1.2, HDAC3sv2, HDAC3sv3, or HDAC3sv4 at a high level. These proteins will be contacted with individual compounds or preparations containing different compounds. A preparation containing different compounds where one or more compounds affect HDAC3sv1.1, HDAC3sv1.2, HDAC3sv2, HDAC3sv3, or HDAC3sv4 in cells over-producing HDAC3sv1.1, HDAC3sv1.2, HDAC3sv2, HDAC3sv3, or HDAC3sv4 as compared to control cells containing expression vector lacking HDAC3sv1.1, HDAC3sv1.2, HDAC3sv2, HDAC3sv3, or HDAC3sv4 coding sequences, can be divided into smaller groups of compounds to identify the compound(s) affecting HDAC3sv1.1, HDAC3sv1.2, HDAC3sv2, HDAC3sv3, or HDAC3sv4 activity, respectively.

HDAC3sv1.1, HDAC3sv1.2, HDAC3sv2, HDAC3sv3, or HDAC3sv4 functional assays can be performed using recombinantly produced HDAC3sv1.1, HDAC3sv1.2, HDAC3sv2, HDAC3sv3, or HDAC3sv4 present in different environments. Such environments include, for example, cell extracts and purified cell extracts containing HDAC3sv1.1, HDAC3sv1.2, HDAC3sv2, HDAC3sv3, or HDAC3sv4 expressed from recombinant nucleic acid; and the use of a purified HDAC3sv1.1, HDAC3sv1.2, HDAC3sv2, HDAC3sv3, or HDAC3sv4 produced by recombinant means that is introduced into a different environment suitable for measuring histone deacetylase activity.

Modulating HDAC3sv1.1, HDAC3sv1.2, HDAC3sv2, HDAC3sv3, and HDAC3sv4 Expression

HDAC3sv1.1, HDAC3sv1.2, HDAC3sv2, HDAC3sv3, or HDAC3sv4 expression can be modulated as a means for increasing or decreasing HDAC3sv1.1, HDAC3sv1.2, HDAC3sv2, HDAC3sv3, or HDAC3sv4 activity, respectively. Such modulation includes inhibiting the activity of nucleic acids encoding the HDAC3 isoform target to reduce HDAC3 isoform protein or polypeptide expressions, or supplying HDAC3 nucleic acids to increase the level of expression of the HDAC3 target polypeptide thereby increasing HDAC3 activity.

Inhibition of HDAC3sv1.1, HDAC3sv1.2, HDAC3sv2, HDAC3sv3, and HDAC3sv4 Activity

HDAC3sv1.1, HDAC3sv1.2, HDAC3sv2, HDAC3sv3, or HDAC3sv4 nucleic acid activity can be inhibited using nucleic acids recognizing HDAC3sv1.1, HDAC3sv1.2, HDAC3sv2, HDAC3sv3, or HDAC3sv4 nucleic acid and affecting the ability of such nucleic acid to be transcribed or translated. Inhibition of HDAC3sv1.1, HDAC3sv1.2, HDAC3sv2, HDAC3sv3, or HDAC3sv4 nucleic acid activity can be used, for example, in target validation studies.

A preferred target for inhibiting HDAC3sv1.1, HDAC3sv1.2, HDAC3sv2, HDAC3sv3, or HDAC3sv4 is mRNA stability and translation. The ability of HDAC3sv1.1, HDAC3sv1.2, HDAC3sv2, HDAC3sv3, or HDAC3sv4 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing HDAC3sv1.1, HDAC3sv1.2, HDAC3sv2, HDAC3sv3, and HDAC3sv4 Expression

Nucleic acids encoding for HDAC3sv1.1, HDAC3sv1.2, HDAC3sv2, HDAC3sv3, or HDAC3sv4 can be used, for example, to cause an increase in HDAC3 activity or to create a test system (e.g., a transgenic animal) for screening for compounds affecting HDAC3sv1.1, HDAC3sv1.2, HDAC3sv2, HDAC3sv3, or HDAC3sv4 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of HDAC3sv1, HDAC3sv2, and HDAC3sv3 Using Microarrays

To identify variants of the “normal” splicing of the exon regions encoding HDAC3, an exon junction microarray, comprising probes complementary to each splice junction resulting from splicing of the 15 exon coding sequences in HDAC3 heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least one of the exon junctions of HDAC3 mRNA was altered in some of the tissues examined, suggesting the presence of HDAC3 splice variant mRNA populations. Reverse transcription and polymerase chain reaction (RT-PCR) were then performed using oligonucleotide primers complementary to exons 1 and 8 to confirm the exon junction array results and to allow the sequence structure of the splice variants to be determined.

### Example 2

### Confirmation of HDAC3sv1, HDAC3sv2, and HDAC3sv3 Using RT-PCR

The structure of HDAC3 mRNA in the region corresponding to exon 1 to 8 was determined for a panel of human tissue and cell line samples using an RT-PCR based assay (FIG. 1). PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 1 and exon 8 of the reference exon coding sequences in HDAC3 (NM_{—}003883). Based upon the nucleotide sequence of HDAC3 mRNA, the HDAC3 exon 1 and exon 8 primer set (hereafter HDAC3₁₋₈ primer set) was expected to amplify a 614 base pairs amplicon representing the “reference” HDAC3 mRNA region. The HDAC3 exon 1 forward primer has the sequence: 5′ CATGGCCAAGACCGTGGCCTATTTCT 3′ [SEQ ID NO 37]; and the HDAC3 exon 8 reverse primer has the sequence: 5′ CACCTGTGCCAGGGAAGAAGTAA TTTCC 3′ [SEQ ID NO 38], wherein the 5′ end of the exon 8 reverse primer is complementary to sequences in exon 8, and the 3′ end of the exon 8 reverse primer spans the exon 7/exon 8 splice junction and is complementary to sequences in exon 7.

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

50° C. for 30 minutes;

95° C. for 15 minutes;

35 cycles of:

94° C. for 1 minute;

60° C. for 1 minute;

72° C. for 1 minute; then

72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least four different RT-PCR amplicons were obtained from human mRNA samples using the HDAC3₁₋₈ primer set (data not shown). Every human tissue and cell line assayed exhibited the expected amplicon size of 614 base pairs for normally spliced HDAC3 mRNA. Except for heart, pancreas, skeletal muscle, and ileocecum, all other human tissue and cell lines assayed also exhibited an amplicon of 276 base pairs in addition to the expected HDAC3 amplicon of 614 base pairs. Except for heart, salivary gland, brain-cerebellum, trachea, thyroid, brain-amygdala, brain-corpus callosum, fetal lung, melanoma, adrenal-medulla, duodenum, and ileum, all other human tissue and cell lines assayed also exhibited an amplicon of 389 base pairs in addition to the expected HDAC3 amplicon of 614 base pairs. Every human tissue and cell line assayed also exhibited an amplicon of about 471 base pairs in addition to the expected HDAC3 amplicon of 614 base pairs. The tissues in which HDAC3sv1, HDAC3sv2 and HDAC3sv3 mRNAs were detected are listed in Table 1.

TABLE 1

| Sample | HDAC3sv1 | HDAC3sv2 | HDAC3sv3 |
|---|---|---|---|
| Heart | | | x |
| Kidney | x | x | x |
| Liver | x | x | x |
| Brain | x | x | x |
| Placenta | x | x | x |
| Lung | x | x | x |
| Fetal Brian | x | x | x |
| Leukemia Promyelocytic | x | x | x |
| (HL-60) | | | |
| Adrenal Gland | x | x | x |
| Fetal Liver | x | x | x |
| Salivary Gland | x | | x |
| Pancreas | | x | x |
| Skeletal Muscle | | x | x |
| Brain Cerebellum | x | | x |
| Stomach | x | x | x |
| Trachea | x | | x |
| Thyroid | x | | x |
| Bone Marrow | x | x | x |
| Brain Amygdala | x | | x |
| Brain Caudate Nucleus | x | x | x |
| Brain Corpus Callosum | x | | x |
| Ileocecum | | x | x |
| Lymphoma Burkitt's (Raji) | x | x | x |
| Spinal Cord | x | x | x |
| Lymph Node | x | x | x |
| Fetal Kidney | x | x | x |
| Uterus | x | x | x |
| Spleen | x | x | x |
| Brain Thalamus | x | x | x |
| Fetal Lung | x | | x |
| Testis | x | x | x |
| Melanoma (G361) | x | | x |
| Lung Carcinoma (A549) | x | x | x |
| Adrenal Medula, normal | x | | x |
| Brain, Cerebral Cortex, | x | x | x |
| normal; | | | |
| Descending Colon, normal | x | x | x |
| Prostate | x | x | x |
| Duodenum, normal | x | | x |
| Epididymus, normal | x | x | x |
| Brain, Hippocamus, normal | x | x | x |
| Ileum, normal | x | | x |
| Interventricular Septum, | x | x | x |
| normal | | | |
| Jejunum, normal | x | x | x |
| Rectum, normal | x | x | x |

Sequence analysis of the about 276 base pair amplicon, herein referred to as “HDAC3sv1,” revealed that this amplicon form results from the splicing of exon 2 of the HDAC3 hnRNA to exon 7; that is, the exon 3, 4, 5, and 6 coding sequences are completely absent. Sequence analysis of the about 389 base pair amplicon, herein referred to as “HDAC3sv2,” revealed that this amplicon form results from the splicing of exon 2 of the HDAC3 hnRNA to exon 5; that is, the exon 3 and 4 coding sequences are completely absent. Sequence analysis of the about 471 base pair amplicon, herein referred to as “HDAC3sv3,” revealed that this amplicon form results from the splicing of exon 2 of the HDAC3 hnRNA to exon 4; that is, the exon 3 coding sequence is completely absent. Thus, the RT-PCR results confirmed the junction probe microarray data reported in Example 1, which suggested that HDAC3 mRNA is composed of a mixed population of molecules wherein in at least one of the HDAC3 mRNA splice junctions is altered.

### Example 3

### Cloning of HDAC3sv2, HDAC3sv4, HDAC3sv5, and HDAC3sv6

Clones having nucleotide sequence comprising the splice variants referred to herein as HDAC3sv4, HDAC3sv5, and HDAC3sv6, and a clone having a partial nucleotide sequence of the splice variant referred to herein as HDAC3sv2, were isolated from commercial cDNA clone libraries (Invitrogen Corporation, Carlsbad, Calif.). A BLAST search of an Invitrogen library database containing only end sequences of the cDNA inserts of each clone in the Invitrogen libraries was performed using the nucleotide sequence of the HDAC3 reference mRNA NM_{—}003883. Thus, a series of new cDNA clones were identified that had end sequences homologous to the HDAC3 reference sequence. RT-PCR was performed on the identified clones using appropriate PCR primers designed to flank the HDAC3 variant splice junctions identified by microarrays (see Example 1).

Clones that yielded a PCR amplicon of a size different than the size expected from amplification of a reference HDAC3 clone were identified. The full-length sequence of the cDNA clones of interest were obtained by primer walking. The variant cDNA clones HDAC3sv4, HDAC3sv5, and HDAC3sv6, and partial HDAC3sv2 were identified by aligning the full-length cDNA clone sequences with the reference sequence NM_{—}003883.

The polynucleotide sequence of partial HDAC3sv2 mRNA (SEQ ID NO 39) contains an open reading frame that encodes a partial HDAC3sv2 protein (SEQ ID NO 40) similar to the reference HDAC3 protein (NP_{—}003874), but lacking the 75 amino acids encoded by a 225 base pair region corresponding to exons 3 and 4, as well as the 114 amino acids encoded by a 342 base pair region corresponding to the last 8 nucleotides of exon 12 and the nucleotides of exons 13, 14, and 15 of the full length coding sequence of reference HDAC3 mRNA (NM_{—}003883). The deletion of the 225 base pair exon 3 to exon 4 region results in a protein translation reading frame that is in alignment in comparison to the reference HDAC3 protein reading frame.

The polynucleotide sequence of HDAC3sv4 (SEQ ID NO 9) contains an open reading frame that encodes a HDAC3sv4 protein (SEQ ID NO 10) similar to the reference HDAC3 protein (NP_{—}003874) but lacking the amino acids encoded by the nucleotides corresponding to exons 5-15 of the full length coding sequence of reference HDAC3 mRNA (NM_{—}003883). HDAC3sv4 mRNA retains intron 4 sequence, resulting in a protein reading frame shift at the novel exon 4/intron 4 splice junction, and creating a protein translation reading frame that is out of alignment in comparison to the reference HDAC3 protein reading frame. The retention of intron 4 and shift in reading frame creates two new amino acids at the carboxy-terminus of the HDAC3sv4 protein and a premature termination codon, resulting in the production of an altered and shorter HDAC3sv4 protein as compared to the reference HDAC3 protein (NP_{—}003874).

The polynucleotide sequence of HDAC3sv5 (SEQ ID NO 20) contains an open reading frame that encodes a HDAC3sv4 protein (SEQ ID NO 10) similar to the reference HDAC3 protein (NP_{—}003874) but lacking the amino acids encoded by the nucleotides corresponding to exons 5-15 of the full length coding sequence of reference HDAC3 mRNA (NM_{—}003883). HDAC3sv5 mRNA retains intron 4 sequence and intron 5 sequence, resulting in a protein reading frame shift at the novel exon 4/intron 4 splice junction, and creating a protein translation reading frame that is out of alignment in comparison to the reference HDAC3 protein reading frame. The retention of intron 4 and shift in reading frame creates two new amino acids and a premature termination codon, resulting in the production of an altered and shorter HDAC3sv4 protein as compared to the reference HDAC3 protein (NP_{—}003874).

The polynucleotide sequence of HDAC3sv6 (SEQ ID NO 21) contains an open reading frame that encodes a HDAC3sv3 protein (SEQ ID NO 8) similar to the reference HDAC3 protein (NP_{—}003874) but lacking the amino acids encoded by the nucleotides corresponding to exons 3-15 of the full length coding sequence of reference HDAC3 mRNA (NM_{—}003883). HDAC3sv6 mRNA deletes exons 3, 10, and 12 coding sequence and retains intron 5 sequence, resulting in a protein reading frame shift at the novel exon 2/exon 4 splice junction, and creating a protein translation reading frame that is out of alignment in comparison to the reference HDAC3 protein reading frame. The deletion of exon 3 and shift in reading frame creates seven new amino acids and a premature termination codon, resulting in the production of an altered and shorter HDAC3sv3 protein as compared to the reference HDAC3 protein (NP_{—}003874).

### Example 4

### Cloning of HDAC3sv1.1, HDAC3sv1.2, HDAC3sv2, and HDAC3sv3

Microarray and RT-PCR data indicate that in addition to the normal HDAC3 reference mRNA sequence, NM_{—}003883, encoding HDAC3 protein, NP_{—}003874, three novel splice variant forms of HDAC3 mRNA also exist in many tissues.

Clones having nucleotide sequence comprising the splice variants identified in Example 2 (hereafter referred to as HDAC3sv1.1, HDAC3sv2, or HDAC3sv3) are isolated using a 5′ “forward” HDAC3 primer and a 3′ “reverse” HDAC3 primer, to amplify and clone the entire HDAC3sv1.1, HDAC3sv2, or HDAC3sv3 mRNA coding sequences, respectively. The same 5′ “forward” primer is designed for isolation of full length clones corresponding to the HDAC3sv1.1, HDAC3sv2, and HDAC3sv3 splice variants and has the nucleotide sequence of 5′ ATGGCCAAGACCGTGGCCTATTTCTAC 3′ [SEQ ID NO 41]. The 3′ “reverse” HDAC3sv1.1 primer is designed to have the nucleotide sequence of 5′ TCAATGTAGAGCA CCCGAGGGTGGTAC 3′ [SEQ ID NO 42]. The 3′ “reverse” HDAC3sv2 primer is designed to have the nucleotide sequence of 5′ TTAAATCTCCACATCGCTTTCCTTGTC 3′ [SEQ ID NO 43]. The 3′ “reverse” HDAC3sv3 primer is designed to have the nucleotide sequence of 5′ TCA AAGAGCCCGGGAAACACTGGGGAT 3′ [SEQ ID NO 44].

RT-PCR

The HDAC3sv1.1, HDAC3sv2, and HDAC3sv3 cDNA sequences are cloned using a combination of reverse transcription (RT) and polymerase chain reaction (PCR). More specifically, about 25 ng of testis polyA mRNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the HDAC3 “forward” and “reverse” primers. After an initial 94° C. denaturation of 1 minute, 35 cycles of amplification are performed using a 30 second denaturation at 94° C. followed by a 1 minute annealing at 65° C. and a 90 second synthesis at 68° C. The 35 cycles of PCR are followed by a 7 minute extension at 68° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted HDAC3sv1.1, HDAC3sv2, and HDAC3sv3 mRNAs, products of about 196, 1093, and 199 bases, respectively. The remainder of the 50 μl PCR reactions from human testis is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC 110A, from Savant, Holbrook, N.Y.) attached to a Universal Vacuum Sytem 400 (also from Savant) for about 30 minutes on medium heat.

Cloning of RT-PCR Products

About 4 Tl of the 6 Tl of purified HDAC3sv1.1, HDAC3sv2, and HDAC3sv3 RT-PCR products from human testis are used in a cloning reaction using the reagents and instructions provided with the TOPO TA cloning kit (Invitrogen, Carlsbad, Calif.). About 2 Tl of the cloning reaction is used following the manufacturer's instructions to transform TOP10 chemically competent E. coli provided with the cloning kit. After the 1 hour recovery of the cells in SOC medium (provided with the TOPO TA cloning kit), 200 Tl of the mixture is plated on LB medium plates (Sambrook, et al., in Molecular Cloning, A Laboratory Manual, 2^{nd} Edition, Cold Spring Harbor Laboratory Press, 1989) containing 100 Tg/ml Ampicillin (Sigma, St. Louis, Mo.) and 80 Tg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative HDAC3sv1.1, HDAC3sv2, and HDAC3sv3 clones, respectively are identified and prepared for a PCR reaction to confirm the presence of the expected HDAC3sv1.1 exon 2 to exon 7, HDAC3sv2 exon 2 to exon 5 and HDAC3 exon 2 to exon 4 splice variant structures. A 25 Tl PCR reaction is performed as described above (RT-PCR section) to detect the presence of HDAC3sv1.1, except that the reaction includes miniprep DNA from the TOPO TA/HDAC3sv1.1 ligation as a template. An additional 25 Tl PCR reaction is performed as described above (RT-PCR section) to detect the presence of HDAC3sv2, except that the reaction includes miniprep DNA from the TOPO TA/HDAC3sv2 ligation as a template. An additional 25 Tl PCR reaction is performed as described above (RT-PCR section) to detect the presence of HDAC3sv3, except that the reaction includes miniprep DNA from the TOPO TA/HDAC3sv3 ligation as a template. About 10 Tl of each 25 Tl PCR reaction is run on a 1% Agarose gel and the DNA bands generated by the PCR reaction are visualized and photographed on a UV light box to determine which minipreps samples have PCR product of the size predicted for the corresponding HDAC3sv1.1, HDAC3sv2, and HDAC3sv3 splice variant mRNAs.

Clones having the HDAC3sv1.1 structure are identified based upon amplification of an amplicon band of 165 basepairs, whereas a normal reference HDAC3 clone will give rise to an amplicon band of 503 basepairs. Clones having the HDAC3sv2 structure are identified based upon amplification of an amplicon band of 1062 basepairs, whereas a normal reference HDAC3 clone would give rise to an amplicon band of 1287 basepairs. Clones having the HDAC3sv3 structure are identified based upon amplification of an amplicon band of 162 basepairs, whereas a normal reference HDAC3 clone would give rise to an amplicon band of 305 basepairs. DNA sequence analysis of the HDAC3sv1.1, HDAC3sv2, or HDAC3sv3 cloned DNAs confirm a polynucleotide sequence representing the deletion of exons 3, 4, 5, and 6 in the case of HDAC3sv1.1; the deletion of exons 3 and 4 in the case of HDAC3sv2; or the deletion of exon 3 in the case of HDAC3sv3.

The polynucleotide sequence of HDAC3sv1 mRNA contains two open reading frames that encode an amino terminal and a carboxy terminal protein, referred to herein as HDAC3sv1.1 and HDAC3sv1.2, respectively. SEQ ID NO 1 encodes the amino terminal HDAC3sv1.1 protein (SEQ ID NO 2), similar to the reference HDAC3 protein (NP_{—}003874), but lacking the amino acids encoded by a 338 base pair region corresponding to exons 3, 4, 5 and 6 of the full length coding sequence of reference HDAC3 mRNA (NM_{—}003883). The alternative spliced HDAC3sv1 mRNA not only deletes a 338 base pair region corresponding to exons 3, 4, 5 and 6, but also results in a protein reading frame shift at the exon 2/exon 7 splice junction, creating a protein translation reading frame that is out of alignment in comparison to the reference HDAC3 protein reading frame. This shift in reading frame creates a premature termination codon, resulting in the production of an altered and shorter HDAC3sv1.1 protein as compared to the reference HDAC3 protein (NP_{—}003874). HDAC3sv1.2 polynucleotide (SEQ ID NO 3) encodes the carboxy terminal HDAC3sv1.2 protein (SEQ ID NO 4), similar to the reference HDAC3 protein (NP_{—}003874), but lacking the first 187 amino acids of the reference HDAC3 protein (NP_{—}003874) due to utilization of a translation initiation AUG codon downstream from the AUG initiation codon utilized by the reference HDAC3 protein (NP_{—}003874).

The polynucleotide sequence of HDAC3sv2 mRNA (SEQ ID NO 5) contains an open reading frame that encodes a HDAC3sv2 protein (SEQ ID NO 6) similar to the reference HDAC3 protein (NP_{—}003874), but lacking the 75 amino acids encoded by a 225 base pair region corresponding to exons 3 and 4 of the full length coding sequence of reference HDAC3 mRNA (NM_{—}003883). The deletion of the 225 base pair region results in a protein translation reading frame that is in alignment in comparison to the reference HDAC3 protein reading frame. Therefore the HDAC3sv2 protein is only missing an internal 75 amino acid region as compared to the reference HDAC3 (NP_{—}003874).

The polynucleotide sequence of HDAC3sv3 mRNA (SEQ ID NO 7) contains an open reading frame that encodes a HDAC3sv3 protein (SEQ ID NO 8) similar to the reference HDAC3 protein (NP_{—}003874), but lacking amino acids encoded by exon 3 of the full length coding sequence of reference HDAC3 mRNA (NM_{—}003883). The alternative splicing of exon 2 to exon 4 not only deletes a 143 base pair region corresponding to exon 3, but also results in a protein reading frame shift at the novel exon 2/exon 4 splice junction, creating a protein translation reading frame that is out of alignment in comparison to the reference HDAC3 protein reading frame. This shift in reading frame creates a premature termination codon, resulting in the production of an altered and shorter HDAC3sv3 protein as compared to the reference HDAC3 protein (NP_{—}003874).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 5, or the complement thereof.

2. The purified nucleic acid of claim 1, wherein said nucleic acid comprises a region encoding SEQ ID NO 6.

3. The purified nucleic acid of claim 1, wherein said nucleotide sequence encodes a polypeptide consisting of SEQ ID NO 6.

4. A purified polypeptide comprising SEQ ID NO 6.

5. The polypeptide of claim 4, wherein said polypeptide consists of SEQ ID NO 6.

6. An expression vector comprising a nucleotide sequence encoding SEQ ID NO 6, wherein said nucleotide sequence is transcriptionally coupled to an exogenous promoter.

7. The expression vector of claim 6, wherein said nucleotide sequence encodes a polypeptide consisting of SEQ ID NO 6.

8. The expression vector of claim 6, wherein said nucleotide sequence comprises SEQ ID NO 5.

9. The expression vector of claim 6, wherein said nucleotide sequence consists of SEQ ID NO 5.

10. A method of screening for compounds able to bind selectively to HDAC3sv2 comprising the steps of:
(a) providing a HDAC3sv2 polypeptide comprising SEQ ID NO 6;
(b) providing one or more HDAC3 isoform polypeptides that are not HDAC3sv2;
(c) contacting said HDAC3sv2 polypeptide and said HDAC3 isoform polypeptide that is not HDAC3sv2 with a test preparation comprising one or more compounds; and
(d) determining the binding of said test preparation to said HDAC3sv2 polypeptide and to said HDAC3 isoform polypeptide that is not HDAC3sv2, wherein a test preparation that binds to said HDAC3sv2 polypeptide, but does not bind to said HDAC3 polypeptide that is not HDAC3sv2, contains a compound that selectively binds said HDAC3sv2 polypeptide.

11. The method of claim 10, wherein said HDAC3sv2 polypeptide is obtained by expression of said polypeptide from an expression vector comprising a polynucleotide encoding SEQ ID NO 6.

12. The method of claim 11, wherein said polypeptide consists of SEQ ID NO 6.

13. A method for screening for a compound able to bind to or interact with a HDAC3sv2 protein or a fragment thereof comprising the steps of:
(a) expressing a HDAC3sv2 polypeptide comprising SEQ ID NO 6 or fragment thereof from a recombinant nucleic acid;
(b) providing to said polypeptide a labeled HDAC3 ligand that binds to said polypeptide and a test preparation comprising one or more compounds; and
(c) measuring the effect of said test preparation on binding of said labeled HDAC3 ligand to said polypeptide, wherein a test preparation that alters the binding of said labeled HDAC3 ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said HDAC3sv2 ligand is an HDAC inhibitor.

18. The method of claim 16, wherein said expression vector comprises SEQ ID NO 5 or a fragment of SEQ ID NO 5.

19. The method of claim 13, wherein said polypeptide comprises SEQ ID NO 6 or a fragment of SEQ ID NO 6.

20. The method of claim 13, wherein said test preparation contains one compound.


SEQUENCE LISTING (44 sequences)

SEQ ID NO: 1 - DNA, 162 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatcgtaccaccctcgggtgctctacat

SEQ ID NO: 2 - protein, 54 aa, Homo sapiens
MAKTVAYFYDPDVGNFHYGAGHPMKPHRLALTHSLVLHYGLYKKMIVPPSGALH

SEQ ID NO: 3 - DNA, 723 bp, Homo sapiens
atgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatt

SEQ ID NO: 4 - protein, 241 aa, Homo sapiens
MTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRPEAPNEFYDGDHDNDKESDVEI

SEQ ID NO: 5 - DNA, 1059 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatcatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatt

SEQ ID NO: 6 - protein, 353 aa, Homo sapiens
MAKTVAYFYDPDVGNFHYGAGHPMKPHRLALTHSLVLHYGLYKKMIICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLLVEEAISEELPYSEYFEYFAPDFTLHPDVSTRIENQNSRQYLDQIRQTIFENLKMLNHAPSVQIHDVPADLLTYDRTDEADAEERGPEENYSRPEAPNEFYDGDHDNDKESDVEI

SEQ ID NO: 7 - DNA, 159 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatccccagtgtttcccgggctctt

SEQ ID NO: 8 - protein, 53 aa, Homo sapiens
MAKTVAYFYDPDVGNFHYGAGHPMKPHRLALTHSLVLHYGLYKKMIPSVSRAL

SEQ ID NO: 9 - DNA, 1411 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccagcatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaaggtgacatagtcccgagtcctgttcttcctttcctctggatccctggactcgggatttaaccctgatcctgggctcccagcttgaggggtgggcaggaaggactgtgacttaggtgtttgtctttcagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatt

SEQ ID NO: 10 - protein, 123 aa, Homo sapiens
MAKTVAYFYDPDVGNFHYGAGHPMKPHRLALTHSLVLHYGLYKKMIVFKPYQASQHDMCRFHSEDYIDFLQRVSPTNMQGFTKSLNAFNVGDDCPVFPGLFEFCSRYTGASLQGATQLNNKVT

SEQ ID NO: 11 - DNA, 40 bp, Homo sapiens
gtctctataagaagatgatcgtaccaccctcgggtgctct

SEQ ID NO: 12 - DNA, 40 bp, Homo sapiens
gtctctataagaagatgatcatctgtgatattgccattaa

SEQ ID NO: 13 - DNA, 40 bp, Homo sapiens
gtctctataagaagatgatccccagtgtttcccgggctct

SEQ ID NO: 14 - DNA, 40 bp, Homo sapiens
caacccagctgaacaacaaggtgacatagtcccgagtcct

SEQ ID NO: 15 - DNA, 40 bp, Homo sapiens
cttaggtgtttgtctttcagatctgtgatattgccattaa

SEQ ID NO: 16 - DNA, 40 bp, Homo sapiens
accatgccaagaagtttgaggtgagtgaggaggtgatggg

SEQ ID NO: 17 - DNA, 40 bp, Homo sapiens
agaccactgtcttgccataggcctctggcttctgctatgt

SEQ ID NO: 18 - DNA, 40 bp, Homo sapiens
ctcagcatccgagggcatgggacatatgagacatcgctgc

SEQ ID NO: 19 - DNA, 40 bp, Homo sapiens
tgaggagcttccctatagtgtatctggaccagatccgcca

SEQ ID NO: 20 - DNA, 1531 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccagcatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaaggtgacatagtcccgagtcctgttcttcctttcctctggatccctggactcgggatttaaccctgatcctgggctcccagcttgaggggtgggcaggaaggactgtgacttaggtgtttgtctttcagatctgtgatattgccattaactgggctggtggtctgcaccatgccaagaagtttgaggtgagtgaggaggtgatgggaaagacagtggccatcctagggtaggtgtttaggatgatggtggggggcagctgggaggggaattgctcttctctttatgagaccactgtcttgccataggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatt

SEQ ID NO: 21 - DNA, 1091 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatccccagtgtttcccgggctctttgagttctgctcgcgttacacaggcgcatctctgcaaggagcaacccagctgaacaacaagatctgtgatattgccattaactgggctggtggtctgcaccatgccaagaagtttgaggtgagtgaggaggtgatgggaaagacagtggccatcctagggtaggtgtttaggatgatggtggggggcagctgggaggggaattgctcttctctttatgagaccactgtcttgccataggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatgggacatatgagacatcgctgctggtagaagaggccattagtgaggagcttccctatagtgtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatt

SEQ ID NO: 22 - DNA, 20 bp, Homo sapiens
gaagatgatcgtaccaccct

SEQ ID NO: 23 - DNA, 20 bp, Homo sapiens
gaagatgatcatctgtgata

SEQ ID NO: 24 - DNA, 20 bp, Homo sapiens
gaagatgatccccagtgttt

SEQ ID NO: 25 - DNA, 20 bp, Homo sapiens
gaacaacaaggtgacatagt

SEQ ID NO: 26 - DNA, 20 bp, Homo sapiens
tgtctttcagatctgtgata

SEQ ID NO: 27 - DNA, 20 bp, Homo sapiens
gaagtttgaggtgagtgagg

SEQ ID NO: 28 - DNA, 20 bp, Homo sapiens
cttgccataggcctctggct

SEQ ID NO: 29 - DNA, 20 bp, Homo sapiens
gagggcatgggacatatgag

SEQ ID NO: 30 - DNA, 20 bp, Homo sapiens
ccctatagtgtatctggacc

SEQ ID NO: 31 - DNA, 20 bp, Homo sapiens
atgacggtgtccttccacaa

SEQ ID NO: 32 - protein, 10 aa, Homo sapiens
YKKMIVPPSG

SEQ ID NO: 33 - protein, 10 aa, Homo sapiens
MTVSFHKYGN

SEQ ID NO: 34 - protein, 10 aa, Homo sapiens
YKKMIICDIA

SEQ ID NO: 35 - protein, 10 aa, Homo sapiens
YKKMIPSVSR

SEQ ID NO: 36 - protein, 10 aa, Homo sapiens
GATQLNNKVT

SEQ ID NO: 37 - DNA, 26 bp, Homo sapiens
catggccaagaccgtggcctatttct

SEQ ID NO: 38 - DNA, 28 bp, Homo sapiens
cacctgtgccagggaagaagtaatttcc

SEQ ID NO: 39 - DNA, 717 bp, Homo sapiens
atggccaagaccgtggcctatttctacgaccccgacgtgggcaacttccactacggagctggacaccctatgaagccccatcgcctggcattgacccatagcctggtcctgcattacggtctctataagaagatgatcatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctg

SEQ ID NO: 40 - protein, 239 aa, Homo sapiens
MAKTVAYFYDPDVGNFHYGAGHPMKPHRLALTHSLVLHYGLYKKMIICDIAINWAGGLHHAKKFEASGFCYVNDIVIGILELLKYHPRVLYIDIDIHHGDGVQEAFYLTDRVMTVSFHKYGNYFFPGTGDMYEVGAESGRYYCLNVPLRDGIDDQSYKHLFQPVINQVVDFYQPTCIVLQCGADSLGCDRLGCFNLSIRGHGECVEYVKSFNIPLLVLGGGGYTVRNVARCWTYETSLL

SEQ ID NO: 41 - DNA, 27 bp, Homo sapiens
atggccaagaccgtggcctatttctac

SEQ ID NO: 42 - DNA, 27 bp, Homo sapiens
tcaatgtagagcacccgagggtggtac

SEQ ID NO: 43 - DNA, 27 bp, Homo sapiens
ttaaatctccacatcgctttccttgtc

SEQ ID NO: 44 - DNA, 27 bp, Homo sapiens
tcaaagagcccgggaaacactggggat